(19) United States
(12) Patent Application Publication
(10) Pub. No.: US 2016/0145352 A1
(43) Pub. Date: May 26, 2016

(54) CAMELID SINGLE HEAVY-CHAIN ANTIBODY DIRECTED AGAINST CHROMATIN AND USES OF SAME

(71) Applicants: INSTITUT NATIONAL DE LA RECHERCHE AGRONOMIQUE, Paris Cedex 07 (FR); UNIVERSITE PAUL SABATIER TOULOUSE III, Toulouse (FR)

(72) Inventors: Gladys MIREY, COLOMIERS (FR); Denis JULLIEN, RAMONVILLE SAINT AGNE (FR); Julien VIGNARD, TOULOUSE (FR); Bernard SALLES, TOULOUSE (FR)

(73) Assignees: Institut National de la Recherche Agronomique, Paris Cedex (FR); Universite Paul Sabatier Toulouse III, Toulouse (FR)

(21) Appl. No.: 14/899,695

(22) PCT Filed: Jun. 20, 2014
(86) PCT No.: PCT/EP2014/062996

§ 371 Date: Dec. 18, 2015

(30) Foreign Application Priority Data
Jun. 21, 2013 (FR) 1355941

(51) Int. Cl. C07K 16/44; G01N 33/68

(52) U.S. Cl. CPC C07K 16/44; G01N 33/6875; C07K 2317/569; C07K 2317/22; C07K 2317/76

(57) ABSTRACT

Disclosed is a polypeptide including a single-domain antibody directed against chromatin, derived from a heavy-chain antibody devoid of a camelid light chain (VHH) and capable of binding specifically with a complex of H2A and H2B histones. This polypeptide is particularly suitable for detecting/viewing chromatin in real time, without interfering with the rate of cell proliferation.

The present invention relates to a polypeptide of the type comprising a single-domain antibody derived from a heavy-chain antibody naturally devoid of a light chain (VHH) of a camelid and directed specifically against chromatin, more particularly against some chromatin proteins, and also to a nucleic acid molecule encoding such a polypeptide. The invention also relates to the use of this polypeptide, in particular for detecting chromatin and/or visualizing chromatin in real time without interfering with the cell proliferation rate.

At the current time, the detection in imaging of chromatin can be carried out by three major types of processes, more particularly:
processes using fluorescent molecules which insert into or bind to DNA, for instance 4′,6′-diamidino-2-phenylindole (DAPI). Most of these molecules require the cells to be fixed and permeabilized. Furthermore, they are often classified as carcinogenic, mutagenic and reprotoxic (CMR) and they require restrictive precautions for use;
processes using conventional antibodies directed against chromatin proteins, typically a histone, which, for their part, can only be used on fixed and permeabilized cells;
and processes comprising the ectoptic expression in the target organism of a histone fused to a fluorescent protein. These processes by transgenesis constitute the only approach which makes it possible to monitor chromatin in real time in living cells.

The present invention aims to provide a polyvalent tool which can be used in all these processes, and which makes it possible in particular to effectively detect/visualize chromatin both in vitro and in vivo, both in the form of recombinant protein replacing conventional antibodies, for example in immunofluorescence or Western blotting techniques, and in real-time imaging of living or fixed cells, by non-invasive direct application on the biological material or by the expression of the coding DNA in said cells. An additional objective of the invention is that this tool can be produced easily and inexpensively, in particular in pure form.

At the origin of the invention is a project with quite another purpose, more particularly a project aimed at developing biomarkers of genotoxic effects, specifically the phosphorylation of serine 139 of the H2AX histone. It was discovered by the present inventors that particular polypeptides, comprising the VHH domain of camelid single-stranded heavy-chain antibodies, had the property of interacting specifically with chromatin, and made it possible to achieve the objectives fixed by the present invention. It was in particular discovered by the present inventors, surprisingly, that these polypeptides constituted a tool making it possible to effectively detect chromatin, and in particular: on the one hand, in the form of recombinant fusion protein with a protein domain detectable by immunofluorescence or Western blotting, with an advantageously negligible background noise; and, on the other hand, expressed in target cells, or after penetration into these target cells, without disrupting the progression of the cell cycle thereof so that they allow the real-time visualization of mitotic chromosomes in dividing living cells, without requiring fixing. It was also discovered by the present inventors that the polypeptides having such particularly advantageous properties were specifically directed against a complex of H2A and H2B histones.

Thus, provided according to the present invention is a polypeptide comprising a single-domain antibody directed against chromatin, this antibody being derived from a heavy-chain antibody naturally devoid of a light chain (VHH) of a camelid and being capable of binding specifically to a complex of H2A and H2B histones.

The expression “capable of binding specifically to a complex of H2A and H2B histones” is intended to mean conventionally in itself, the fact that the antibody is only able to bind to the H2A and H2B histones in their heterodimeric form, and not to each of these histones isolated from one another in any other form whatsoever.

The term “complex of H2A and H2B histones” includes herein the H2A-H2B complexes in which one of the H2A and H2B histones, or both, has (have) undergone one or more post-translational modifications, such as an acetylation, methylation, phosphorylation, etc. The expressions “H2A-H2B heterodimer” and “H2A-H2B dimer” will be used, without implied distinction, to denote this complex in the present description.

Single-domain antibodies are natural antibodies well known in themselves, consisting of heavy-chain antibodies devoid of light chains. Their variable domain commonly called VHH, or “nanobody”, is typically formed from a plurality of regions, including a plurality of conserved framework regions, referred to as FR, and a plurality of hypervariable regions, determining the complementarity with the antigen, referred to as CDR. More specifically, the VHH domain comprises a first framework region FR1, a first hypervariable region CDR1, a second framework region FR2, a second hypervariable region CDR2, a third framework region FR3, a third hypervariable region CDR3, and a fourth framework region FR4.

The VHH domain of camelid single-stranded heavy-chain antibodies is sufficient to recognize the antigen: this monomeric and autonomous domain has antigen-binding properties similar to those of conventional antibodies. It is small (15 kDa), particularly stable and soluble, and has in particular the advantage of being able to be produced recombinantly, in bulk, in bacteria or other species, both prokaryotic and eukaryotic, where appropriate as a fusion with one or more functional protein(s) of interest, and of thus being able to be used as a monoclonal antibody for detecting an antigen.

The expression “antibody derived from a heavy-chain antibody naturally devoid of a light chain (VHH) of a camelid” is intended to mean, in the present description, both the VHHs of camelid themselves, and their derivatives, for example humanized VHHs.

As set out above, it has now been discovered by the present inventors that polypeptides comprising an antibody derived from a VHH domain of a camelid, and capable of binding specifically to an H2A-H2B heterodimer, prove to be particularly effective tools for detecting/visualizing chromatin, by cell imaging, Western blotting, flow cytometry, etc., techniques, and also for monitoring chromatin by real-time cell imaging in living or fixed cells. In the latter case, the polypeptides according to the invention even make it possible to visualize any micronuclei, and also anaphase bridges, characteristic of a mytosis defect. The polypeptides according to the invention also prove to be particularly advantageous for carrying out experiments for functional characterization, inactivation or chemical modification of chromatin.

More particularly, the polypeptides according to the invention, which specifically recognize chromatin, can be used as antibodies to replace conventional antibodies, in particular in biochemistry, for example for carrying out conventional and overlay or pull-down Western blotting techniques, enzyme-linked immunosorbent assay (ELISA) techniques, etc. Compared with the already available technology of conventional antibodies, which consist of two heavy chains and two light chains, these VHH-domain polypeptides prove to be more competitive. Indeed, on the one hand, their monomeric nature makes it possible to easily express them genetically, contrary to conventional IgGs, and on the other hand, their small size gives them cell-penetration but also intramolecular properties which are notable compared with conventional antibodies. They can advantageously be expressed in bacteria, and therefore purified on a large scale and at very low cost. In addition, it has in particular been observed by the present inventors that these polypeptides allow effective detection of chromatin, with an extremely low background noise, this being in all eukaryotes by immunofluorescence.

Furthermore, these polypeptides can be expressed directly in cells and they therefore allow real-time monitoring of chromatin structures, for example for monitoring mytosis on living cells, both in lower eukaryotes and in higher eukaryotes. One of the main advantages compared with the processes based on the ectoptic expression of a tagged histone in the cell, as proposed in the prior art, is the fact that they do not modify the stoichiometry of the targets. They also allow good visualization of chromatin and advantageously do not disrupt the cell cycle.

On fixed cells, the polypeptides according to the invention also constitute an advantageous alternative, in particular from the point of view of the cost of production and use, to the use of intercalating agents for dying the nucleus, such as DAPI or propidium iodide. In addition, they can be simultaneously fused to various functional peptides/proteins of interest, which makes it possible to go from a single excitation/emission condition, for chemical intercalating agents, to several conditions for the polypeptides in accordance with the invention, thus broadening the possibilities of multiple labeling.

Generally, the polypeptides according to the invention can, for example, be produced by immunization of camelids, for example of lamas, with an H2A-H2B heterodimer as immunogen, purification of the lymphocytes and construction of a VHH library, then selection in particular by the “phage-display” technique, from this library, expressed by the capsid of a phage, of the coding sequences of VHH exhibiting affinity for an H2A-H2B heterodimer. These techniques are conventional in themselves, and well known to those skilled in the art. They are in particular described in the publication by Lee et al. (2007). The polypeptides according to the invention can otherwise be obtained by selection, from a naïve library of a camelid repertoire, using the same phage-display technique, of the coding sequences of VHH exhibiting affinity for an H2A-H2B heterodimer.

The selection of the VHHs capable of binding to a heterodimer of H2A and H2B histones can be carried out by any antigen-binding assay conventional in itself. To this effect, it is possible to carry out the techniques, well known to those skilled in the art, of immunoblotting, dot blot, or enzyme-linked immunoabsorbent assay ELISA, by taking advantage of the fact that the H2A-H2B dimer can be formed by simply bringing the two proteins into contact, under appropriate physicochemical conditions, in particular of pH and salinity. To this effect, the human histones H2B and H2A, having respectively the Genbank accession No. NP_733759.1 (GI:24586679) (H2A type 1-A) and NP_003505.1 (GI:4504249) (H2B type 1), can in particular be used. Since the structure of the H2A and H2B histones is very strongly conserved in eukaryotes, the antibodies selected for their capacity to bind to this dimer of human origin also have the capacity to bind to the H2A-H2B dimers of any other eukaryotic species, and also in particular to dimers comprising a variant of H2A.

In humans, the H2A histone belongs to the histones which have the highest number of known variants. In addition to the canonic H2A, the family comprises four major variants encoded by distinct paralogous genes. The ubiquitous variants are the H2AX histone, which plays a central role in the cell response to DNA double-strand breaks, and the H2AZ histone, which is very conserved in eukaryotes, and essential to cell viability. The other two members of the H2A family have specific expression profiles. They are macro H2A, which is on the inactive X chromosome, and H2ABdd, which is testicular- and brain-specific. The polypeptides in accordance with the invention bind with great specificity both to the H2A-H2B dimer and to the other dimers comprising a variant of H2A, in particular H2AX-H2B and H2AZ-H2B.

Particular polypeptides according to the invention are such that the single-domain antibody directed against chromatin has:
the amino acid sequence of any one of the sequences SEQ ID No. 1, SEQ ID No. 2, SEQ ID No. 3 or SEQ ID No. 4,
a sequence comprising one or more deletion(s), addition(s) or substitution(s) of one or more amino acids with respect to one of these sequences, which do not significantly modify binding characteristics of the antibody to the complex of H2A and H2B histones,
and/or a functional portion of one of the above sequences, conserving the binding site(s) and the protein domain(s) required for binding to the complex of H2A and H2B histones.

It falls within the skills of those skilled in the art to determine which modifications can be introduced into the above sequences SEQ ID No. 1, SEQ ID No. 2, SEQ ID No. 3 or SEQ ID No. 4, without significantly impairing the antigen recognition properties, on the basis of theoretical knowledge and/or experimental tests, in particular assays for binding to the H2A-H2B heterodimer antigen, as indicated above, carried out on sequences obtained by targeted or random modifications of the sequences SEQ ID No. 1 to 4 above. The characteristics of binding of the antibody to the complex of H2A and H2B histones, i.e. the avidity and the specificity, are considered herein to be not significantly impaired when the antibody makes it possible, for example, when used in the immunofluorescence imaging technique, as a fusion with a detectable protein domain, to visualize chromatin with a low background noise.

Preferentially, the antibodies of which the sequences comprises one or more deletion(s), addition(s) or substitution(s) of one or more amino acids with respect to one of the sequences SEQ ID No. 1, SEQ ID No. 2, SEQ ID No. 3 or SEQ ID No. 4, and/or which have a functional portion of one of these sequences, which retain the binding site(s) and the protein domain(s) required for binding to the complex of H2A and H2B histones, exhibit a performance level in ELISA of at least 50%, preferably of at least 70%, compared with either of the antibodies of sequences SEQ ID No. 1 or SEQ No. 2.

In particular, the modifications with respect to the sequences SEQ ID Nos 1 to 4 above are preferentially carried out in the CDR hypervariable regions of the VHHs.

Polypeptides obtained by modifications of the sequences SEQ ID Nos 1 to 4 by substitution of amino acids with amino acids of the same family, for example of a basic residue such as arginine with another basic residue such as a lysine residue, of an acid residue such as aspartate with another acid residue such as glutamate, of a polar residue such as serine with another polar residue such as threonine, of an aliphatic residue such as leucine with another aliphatic residue such as isoleucine, etc, fall for example within the scope of the invention.

Some polypeptides according to the invention have a sequence homologous to any one of these above sequences SEQ ID No. 1, SEQ ID No. 2, SEQ ID No. 3 and SEQ ID No. 4, with a sequence identity of 79.5% and more relative to at least one of these sequences.

According to one preferential characteristic of the invention, the single-domain antibody directed against chromatin is a VHH domain.

Alternatively, this single-domain antibody directed against chromatin can consist of a humanized VHH, i.e. a VHH comprising one or more amino acids of the human consensus sequence in place of one or more typical amino acids of camelids. It falls within the competence of those skilled in the art to determine which amino acids can thus be substituted without losing the capacity for binding to the H2A-H2B antigen, as indicated above.

Also falling within the scope of the invention are polypeptides of which the sequence is modified, with respect to the sequences SEQ ID No. 1 to 4 above, so as to increase the affinity more specifically with respect to an H2A-H2B dimer in which one or both histones is (are) modified by post-translational modifications participating, for example, in the response to stress and more generally in the expression of the epigenome. Such polypeptides advantageously make it possible to specifically monitor post-translational modifications of the histones, like conventional antibodies, but with the possibility of obtaining a diversity rapidly generated by molecular biology techniques, screens on modified histones obtained by a biochemical purification and characterization thereof by mass spectrometry. They also make it possible to demonstrate differential patterns, in vivo or after fixing, obtained in response to various cell stresses (endogenous or exogenous), in a use in prognostic or diagnostic biology.

Preferentially, when the single-domain antibody directed against chromatin is a VHH domain, it has one or more of the following characteristics:
in its CDR1 hypervariable region, an arginine residue or a glycine residue in position 29 according to Kabat numbering, i.e. in the first position of the CDR1 region, and/or a serine or threonine residue in position 30 according to Kabat numbering, i.e. in the second position of the CDR1 region;
in its CDR2 hypervariable region, a glycine residue in position 58 according to Kabat numbering, i.e. in the fifth position of the CDR2 region, and/or a threonine residue or a serine residue in the seventh position of the CDR2 region;
and in its CDR3 hypervariable region, an asparagine residue or an aspartic acid residue in the last position of the CDR3, and/or at least one of the following amino acids, at the following respective positions, these positions also being defined according to Kabat numbering:
   in position 107 according to Kabat numbering, an arginine residue;
   and/or in position 104 according to Kabat numbering, a glycine residue;
   and/or in position 105 according to Kabat numbering, a serine residue or a tyrosine residue;
   and/or in position 110 according to Kabat numbering, a serine residue or a threonine residue.

The polypeptide according to the invention may comprise one or more single-domain antibodies directed against chromatin.

In particular embodiments of the invention, the polypeptide comprises a plurality of single-domain antibodies directed against chromatin, each of these antibodies being derived from a heavy-chain antibody naturally devoid of a light chain (VHH) of a camelid and being capable of binding specifically to a complex of H2A and H2B histones. The term “diabody” can be used to describe such a polypeptide comprising two VHH-derived single-domain antibodies.

These VHH-derived single-domain antibodies directed against chromatin can have the same peptide sequence, or different peptide sequences.

Such polypeptides advantageously constitute probes that are less dynamic than the polypeptides containing just one single-domain antibody directed against chromatin, their H2A-H2B-antigen-binding time being extended. These probes prove to be particularly useful for carrying out immunofluorescence techniques, in particular because their antigen binding is particularly resistant to washing. They make it possible in particular to visualize with high sensitivity heterochromatin, despite the very compacted structure of the latter. They also make it possible to broaden the field of visualization of chromatin, for example to visualize genes that have been turned off, and also to differentiate various states of the chromatin.

The polypeptide according to the invention may also comprise a peptide sequence corresponding to a functional peptide of interest or a functional protein of interest, or a plurality of such peptide sequences corresponding to a plurality of functional peptides/proteins of interest. The single-domain antibody directed against chromatin and said functional peptide(s)/protein(s) of interest may be separated by a spacer in the fusion protein thus formed.

According to the invention, the functional peptides/proteins of interest may be of any type, depending on the particular use intended for the polypeptide. For example, but in a nonlimiting manner, they may be proteins that are detectable, preferably proteins that are detectable by fluorescence, luminescence or phosphorescence, intracellular trafficking proteins, or else proteins, in particular enzymes, the effect of which on chromatin or histones it is desired to study, or which it is desired that they affect the epigenetic marks of chromatin. For the application of the polypeptides according to the invention for detecting/visualizing chromatin in real time, nonlimiting examples of such functional proteins of interest are the fluorophores GFP, YFP, mCherry, the HA tag, the SNAP tag, the Flag tag, etc. For application to the study of chromatin-fiber or histones modification, examples of functional proteins of interest which form part of the chimeric polypeptide according to the invention are E3 ubiquitin ligases, histone acetyltransferases, histone methyltransferases, biotin ligases, endonucleases, etc.

The functional peptide(s)/protein(s) of interest can be fused to a single-domain antibody directed against chromatin at the C-terminal end and also at the N-terminal end of said antibody. Preferentially, the fusion is carried out at the C-terminal end of the antibody.

The polypeptide according to the invention may also advantageously comprise a peptide sequence corresponding to a cell-penetrating peptide, referred to as CPP, such as the TAT (transactivator of transcription) peptide of the HIV1 virus, or the Penetratin peptide sold by the company Innovagen (Chiu et al., 2010).

A particular polypeptide according to the invention comprises a peptide sequence corresponding to a cell-penetrating peptide, and a peptide sequence corresponding to a detectable protein, such as a fluorescent protein. These peptide sequences can be fused at the same end, C-terminal or N-terminal, of the single-domain antibody directed against chromatin, or respectively at its two opposite ends. Such a polypeptide proves to be in particular entirely advantageous for an application for labeling chromatin in living cells. To this effect, it can be directly applied, in the form of purified recombinant protein, to the cell culture, noninvasively, and can thus allow the real-time visualization of chromatin, without requiring the use of transgenesis techniques.

Another aspect of the invention is a nucleic acid molecule encoding a polypeptide corresponding to one or more of the characteristics above.

The invention also relates to an expression vector comprising such a nucleic acid molecule. This expression vector may be of any type known in itself for use in genetic engineering, in particular a plasmid, a cosmid, a virus or a bacteriophage, containing the elements required for the transcription and translation of the sequence encoding the polypeptide according to the invention.

An additional subject of the invention is a host cell comprising a nucleic acid molecule encoding a polypeptide according to the invention, and/or an expression vector comprising such a molecule. This host cell can be a prokaryotic cell, in particular a bacterial cell, in particular for the bulk production of the polypeptide in accordance with the invention, and also a lower or higher eukaryotic cell, for example a yeast, invertebrate or mammalian cell. In particular, cell lines stably, inducibly or constitutively or else transiently expressing a polypeptide according to the invention also fall within the scope of the invention.

According to another aspect, the invention relates to a transgenic non-human animal expressing a polypeptide according to the invention, in particular a polypeptide comprising a peptide sequence corresponding to a detectable protein, for example a protein detectable by fluorescence, luminescence or phosphorescence.

Such a transgenic animal proves especially to be particularly useful for applications for medical purposes, in particular:
in the research field, for studying modes of embryonic development in particular for establishing where and when cell divisions occur, how cells move with respect to one another during the development of an organism of interest, etc.;
in the prevention field, for establishing the innocuousness of chemical elements on the basis of the exposure of the transgenic animal models ad hoc optionally during their embryonic development, by real-time monitoring of cell division defects, of nucleic shape defects, of proliferation defects, etc.;
for evaluating compounds of therapeutic interest.

Such a transgenic animal can be obtained by any method conventional in itself, in particular by transformation with a vector comprising a transgene encoding a polypeptide in accordance with the invention. Preferentially, it has integrated this transgene into its genome.

This animal is preferably chosen from invertebrates, such as Caenorhabditis elegans or Drosophila melanogaster, lower vertebrates such as the zebra fish, or mice.

In particular, it has been shown by the present inventors that a transgenic drosophila expressing a polypeptide in accordance with the present invention, comprising a peptide sequence corresponding to a protein detectable by fluorescence, develops normally from the embryonic stage up to the adult stage. The polypeptide specifically labels the chromatin therein, and advantageously remains associated therewith throughout development up to the adult stage.

According to another aspect, the present invention relates to a kit, which has an application in particular for detecting and/or visualizing chromatin in real time, in particular a complex of H2A and H2B histones, or else for the modification thereof, the purification thereof, and more generally for the study thereof. This kit comprises a polypeptide having one or more of the characteristics above, and/or a nucleic acid molecule encoding such a polypeptide, and/or an expression vector comprising such a nucleic acid molecule and/or a host cell comprising them.

The present invention also relates to a process for producing a polypeptide having one or more of the above characteristics, in particular with a view to the use thereof as an antibody, this process comprising:
culturing host cells comprising a nucleic acid molecule encoding a polypeptide according to the invention, under conditions which allow the expression of this polypeptide, for example bacterial cells, such as Escherichia coli, cells of a yeast, such as Saccharomyces cerevisiae, mammalian cells or insect cells,
and recovering the polypeptide thus produced.

Such a process enables in particular, when carried out in Escherichia coli for example, a bulk production of the polypeptide at low cost.

The polypeptides according to the invention, the nucleic acid molecules encoding these polypeptides, the expression vectors comprising these nucleic acid molecules, and the host cells comprising such nucleic acid molecules and/or expression vectors, have many applications, taking advantage both of the properties of affinity of the single-domain antibody for the H2A and H2B histones in their heterodimeric form, and of the properties of the VHH domains of camelid single-stranded heavy-chain antibodies, which properties, when combined, advantageously allow particularly effective targeting of chromatin, both in vitro and in vivo.

The present invention thus relates, generally, to the use of a polypeptide having one or more of the above characteristics, and/or of a nucleic acid molecule encoding this polypeptide, and/or of an expression vector comprising such a nucleic acid molecule, and/or of a host cell comprising them, for binding a functional peptide/protein of interest to chromatin, and more particularly to one or both histone(s) of the H2A-H2B heterodimer, or else for causing functional interference in cellulo by inhibition of the binding of these histones with a natural ligand. In the first case, the type of functional peptide/protein of interest is chosen according to the particular application intended.

In particular, according to the invention, the polypeptides, the nucleic acid molecules, the expression vectors and/or the host cells according to the invention can advantageously be used for detecting or visualizing chromatin, in particular a histone of the H2A-H2B heterodimer, in real time, in cell imaging.

For example, in recombinant protein form, the polypeptides according to the invention, in which the single-domain antibody directed against chromatin is fused to a detectable peptide tag, can be used to replace conventional antibodies, for carrying out detection techniques by immunofluorescence, on fixed cells, or by Western blotting. Entirely advantageously, they then allow chromatin labeling in a single step.

By immunofluorescence, on fixed cells, the polypeptides according to the invention make it possible entirely advantageously to detect chromatin with a negligible background noise, this being in all eukaryotes. They in particular allow imaging of mitotic chromosomes in dividing cells.

When expressed directly, by means of an expression vector in accordance with the invention, in the target cells, whether they are living or fixed, the single-domain antibody directed against chromatin being fused with a detectable peptide tag, for example a protein fluorophore, they also make it possible to carry out real-time imaging of chromatin. Experiments in which eukaryotic cells thus transfected are imaged in real time have in particular revealed a very clear concentration of the protein fluorophore on chromatin in interphase, thereby making it possible to visualize the cell nucleus, and also in particular the micronuclei and the anaphase bridges. This advantageous result is obtained whatever the protein fluorophore used, and whether the fusion of the latter to the single-domain antibody directed against chromatin is carried out at the C-terminal or N-terminal of said antibody. It is thus possible, according to the invention, to monitor in real time all of the choreography of chromosomes from prophase to telophase. Furthermore, the fact that the cells expressing a polypeptide according to the invention enter into mytosis demonstrates that said polypeptide advantageously does not interfere with the progression of the cell cycle when it is expressed in the cell.

Advantage can advantageously be taken of the properties of the polypeptides according to the invention in order to carry out genotoxicity tests. Thus, the present invention also relates to the use of the polypeptides, nucleic acid molecules, expression vectors and/or host cells according to the invention, for visualizing chromatin mitotic profile disruptions on living cells ex vivo, in particular visualizing the appearance of one or more micronuclei, of nuclear fragments, of delayed migration of a chromosome, of anaphase bridges, i.e. of chromatin fibers linking two daughter cells, etc., in particular after bringing into contact with a substance of which the genotoxicity must be evaluated.

In particular, the “micronucleic” test is a regulatory test used for genotoxicity studies, which makes it possible to detect substances which cause cytogenetic lesions, in particular clastogenic substances, which generate DNA breaks, and/or aneugenic substances, which cause an abnormal number of chromosomes in the daughter cells, after mytotis (Kirsch-Volders (1997): OECD Guideline for tests on chemical products, No. 487, July 2010).

On fixed and permeabilized cells, the polypeptides according to the invention, which can be produced at low cost, constitute an advantageous alternative to the use of intercalating agents of DAPI type or other, non-intercalating, dyes which are, for their part, much more expensive and potentially cancerogenic.

When it is produced in recombinant form, chemically associated with, or comprising, as a translational fusion with the single-domain antibody directed against chromatin, a protein fluorophore or an enzyme of interest, and optionally a cell-penetrating peptide, it is possible to advantageously take advantages of the properties of the polypeptide according to the invention to bring the protein fluorophore or the enzyme of interest to the chromatin in living cells. This can be carried out by simply bringing the polypeptide into contact with these cells, following which said polypeptide penetrates into the cells, and spontaneously associates the chromatin. The visualization or the modification of the chromatin in the living cells can then be advantageously carried out within transgenesis, and can be very easy to implement.

One application of the polypeptides according to the invention, in which the single-domain antibody directed against chromatin is fused to a protein detectable by fluorescence, luminescence or phosphorescence, and where appropriate to a cell-penetrating peptide, and which are produced in the recombinant form, is thus the use thereof for visualizing chromatin in real time in living cells, by simply adding the polypeptide to the cell culture medium.

The polypeptide according to the invention and more particularly a stable transgenic cell line which expresses it, or living cells into which it has penetrated, can also be used for studying the cell cycle, the localization of the nucleus and the morphology of the nucleus, this being without the need for fixing or labeling by immunofluorescence.

According to another aspect, the invention relates to the use of a polypeptide having one or more of the above characteristics, and/or of a nucleic acid molecule encoding such a polypeptide, and/or of an expression vector comprising such a nucleic acid molecule, and/or of a host cell comprising them, for modifying chromatin fibers in vitro or ex vivo, on fixed cells or on living cells in culture. Preferentially, the single-domain antibody directed against chromatin is then fused to an enzyme of which the effect on chromatin has to be studied. The invention then entirely advantageously has numerous applications, for example for modifying the overall condition of chromatin and therefore altering cell responses, after various treatments or for screening for candidates for therapeutic purposes.

In recombinant form, the polypeptide according to the invention may also be used for purifying chromatin, and in particular the H2A-H2B dimer, for example by means of an immunoprecipitation technique.

The polypeptide according to the invention has a number of other applications. In particular, in biochemistry, in the recombinant form, fused to an appropriate tag, such as a GST (glutathione-S-transferase) or CBD (chitin-binding domain) tag for example, the polypeptide according to the invention can be immobilized on a solid support, in particular in the form of magnetic beads, sepharose beads, etc. The affinity matrix thus generated can then be used to purify chromatin, to immobilize nucleosomes, to purify the histone octamer, or else the H2A-H2B heterodimer, etc.

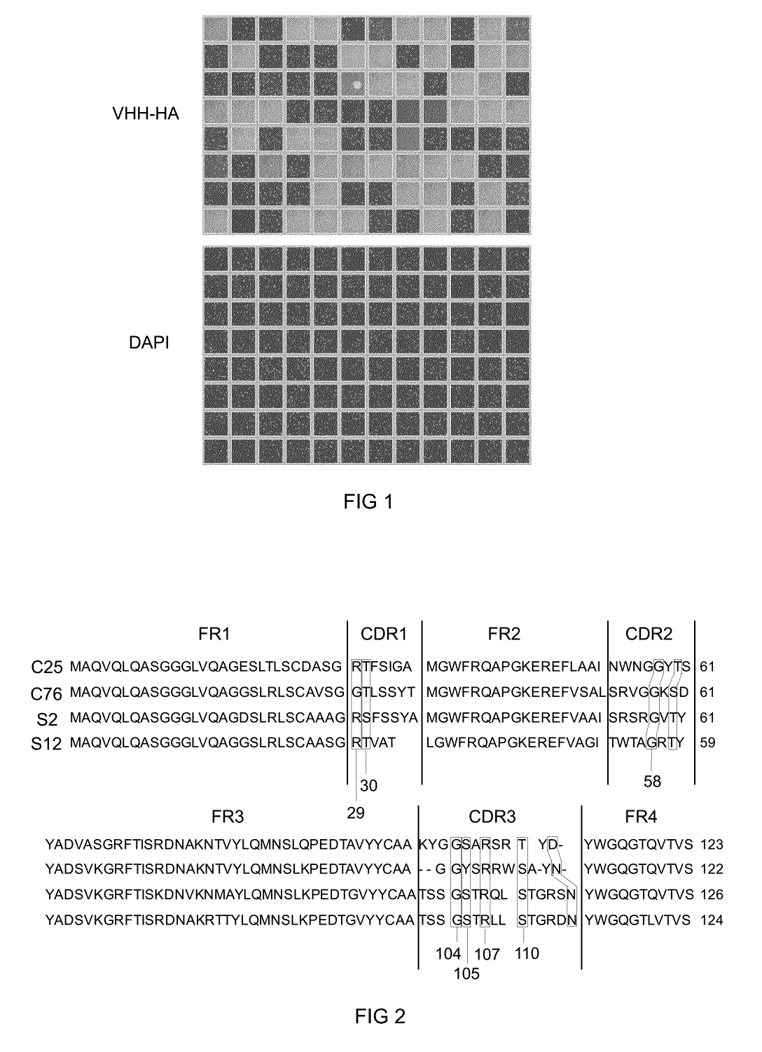

The characteristics and the advantages of the invention will emerge more clearly in the light of the examples hereinafter, provided simply by way of illustration, which is in no way limiting, of the invention, with the support of FIGS. 1 to 26, in which:

FIG. 1 shows, for 96 VHH-HAs derived from a sublibrary of VHH selected by phage display, the images obtained by microscopy after immunofluorescence on fixed cells (top image) and the DAPI signal (bottom image);

FIG. 2 shows the primary structures of four single-domain antibodies directed against chromatin according to the invention, called respectively S2 (of sequence SEQ ID No. 1), S12 (of sequence SEQ ID No. 2), C25 (of sequence SEQ ID No. 3) and C76 (of sequence SEQ ID No. 4); in this figure, the various conserved regions (FR1, FR2, FR3 and FR4) and hypervariable regions (CDR1, CDR2, CDR3) of these antibodies are aligned;

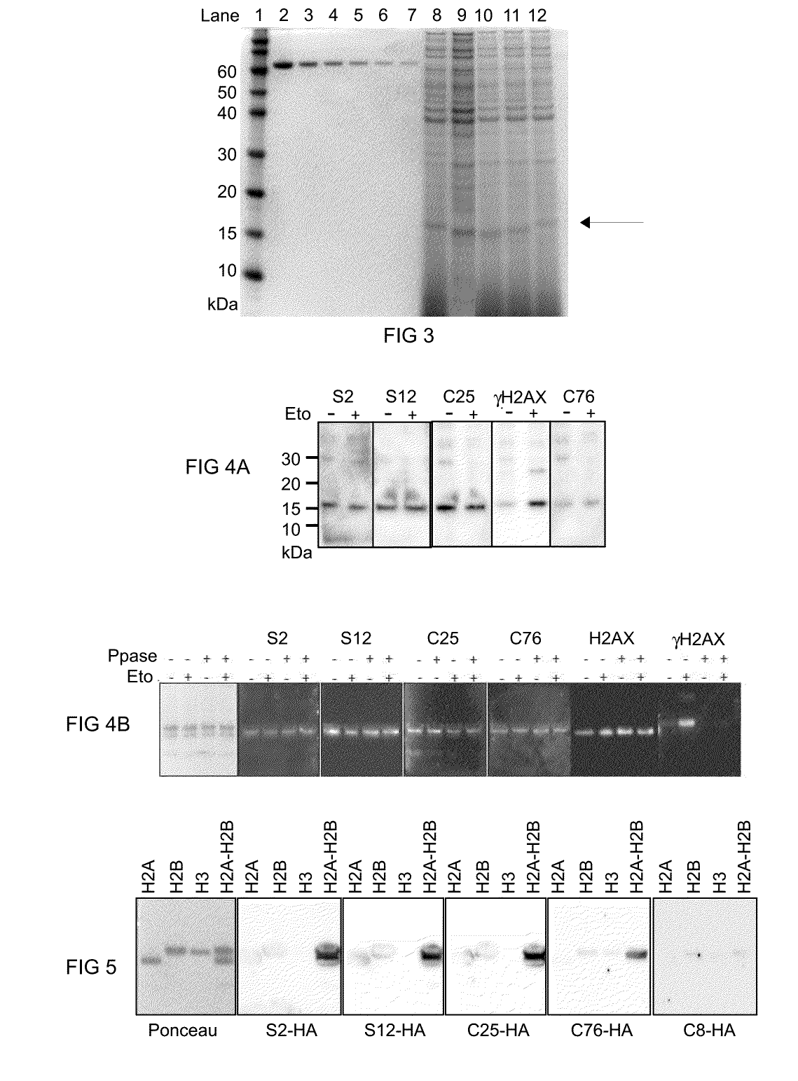

FIG. 3 represents a photograph of a denaturing polyamide gel stained with Coomassie blue, after migration: lane 1, of a molecular weight marker; lanes 2 to 7, of a solution containing respectively 1000, 500, 250, 125, 62 and 31 ng of BSA; lanes 8 to 11, of 8 μl of a bacterial culture medium into which the polypeptides in accordance with the invention, respectively S2-HA, S12-HA, C25-HA and C76-HA, were secreted; lane 12, of 8 μl of a bacterial culture medium into which the comparative polypeptide C8-HA was secreted; the arrow indicates the band corresponding to these polypeptides;

FIGS. 4A and 4B show the results of immunoblotting tests carried out on protein extracts of human cells, by means respectively of S2-HA, S12-HA, C25-HA and C76-HA polypeptides in accordance with the invention, and of anti-H2AX and anti-γH2AX monoclonal antibodies; FIG. 4A, for total protein extracts of HCT116 human cells, treated (“Eto+”) or not treated (“Eto−”) with the genotoxic agent etoposide, the molecular weight marker being represented on the left in the figure; FIG. 4B, for the total histones, obtained by acid extraction from HT1080 human cells (treated (“Eto+”) or not treated (“Eto−”) with the genotoxic agent etoposide), then subjected (“Ppase+”) or not subjected (“Ppase−”) to dephosphorylation, the image on the left corresponding to a membrane stained with Ponceau red;

FIG. 5 shows the results of immunoblotting tests carried out on solutions of pure histones H2A, H2B and H3 and of an H2A-H2B mixture, by means respectively of S2-HA, S12-HA, C25-HA and C76-HA polypeptides in accordance with the invention, and of the comparative polypeptide C8-HA; the image on the left corresponds to a membrane stained with Ponceau red before incubation with the polypeptides;

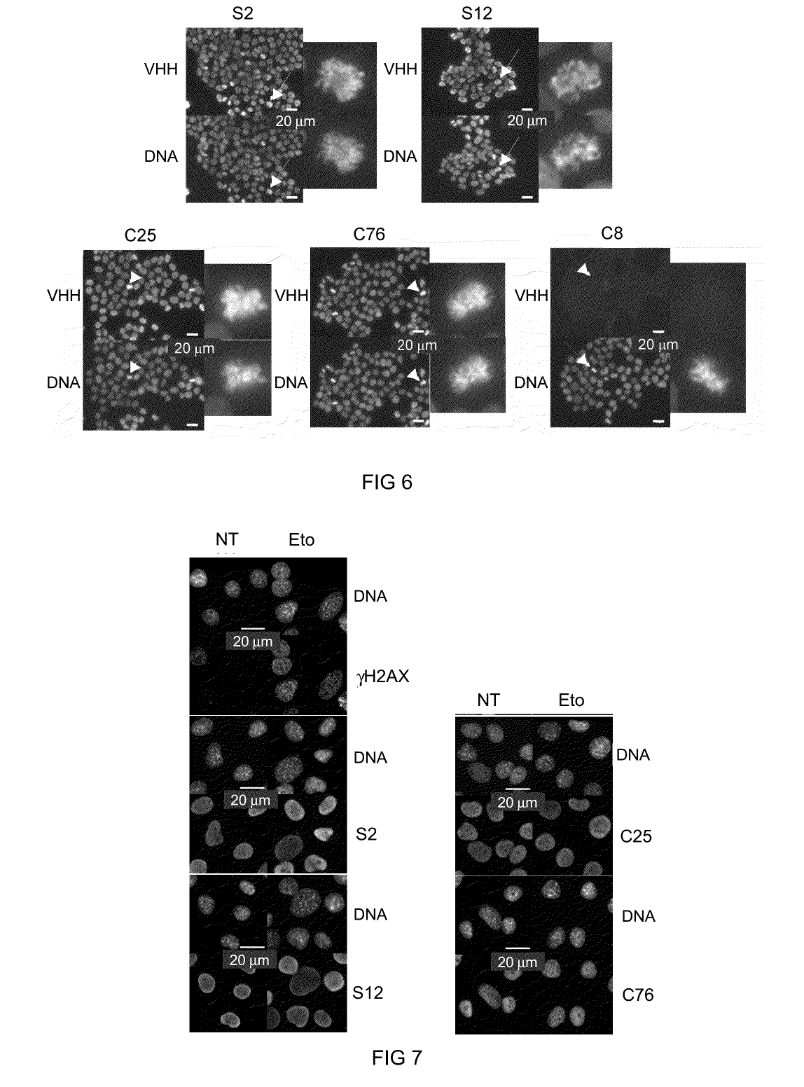

FIG. 6 shows the fluorescence microscopy images of fixed and permeabilized HCT116 human cells processed by immunofluorescence, showing the VHH signal (image top left) and the DAPI signal (image bottom left), for polypeptides according to the invention (S2-HA, S12-HA, C25-HA and C76-HA) and for a comparative polypeptide C8-HA; for each image, the associated image on the right shows a magnification of a cell in mytosis indicated by the white arrow on the image on the left;

FIG. 7 shows fluorescence microscopy images of wild-type murin embryonic fibroblasts, on which DNA damage has been induced (“Eto”) or has not been induced (“NT”), processed by immunofluorescence with polypeptides according to the invention (S2-HA, S12-HA, C25-HA and C76-HA), and an anti-γH2AX antibody (control); for each one, on the top image, “DNA” represents the signal emitted by the DAPI;

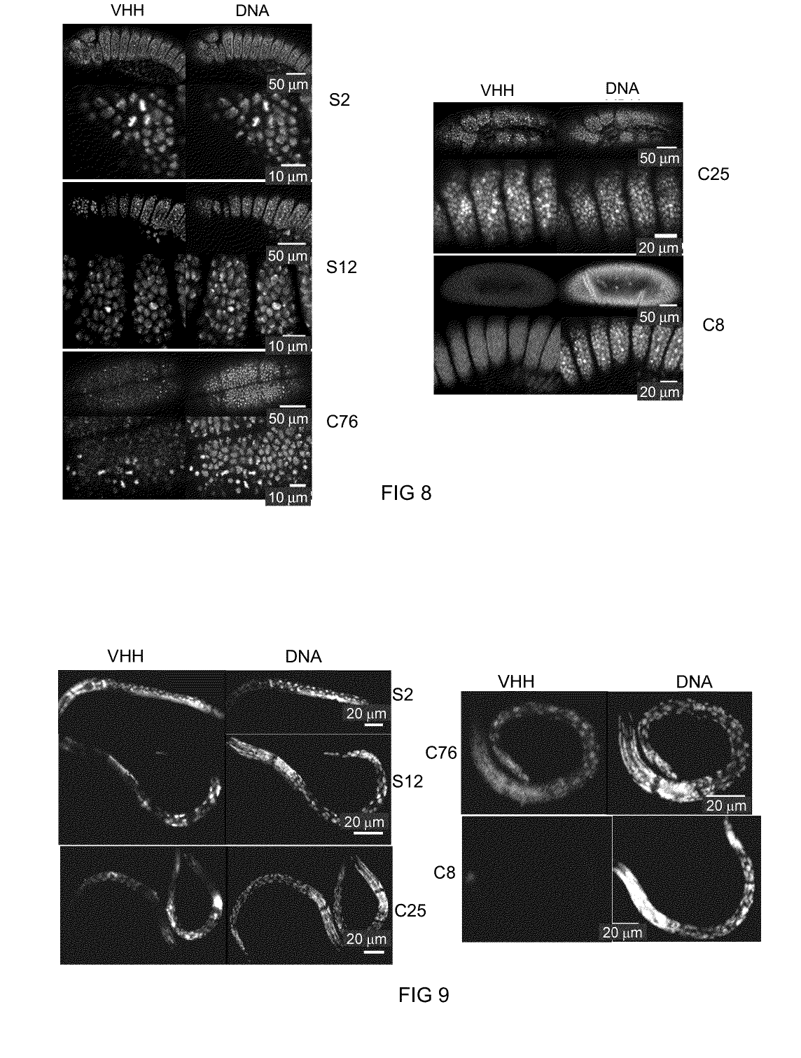

FIG. 8 shows fluorescence confocal microscopy images of Drosophila melanogaster embryos, fixed and processed by immunofluorescence with polypeptides according to the invention (S2-HA, S12-HA, C76-HA and C25-HA) and a comparative polypeptide C8-HA showing, for each one of these polypeptides, the VHH signal (image on the left) and the DNA signal revealed by staining with propidium iodide (image on the right);

FIG. 9 shows fluorescence confocal microscopy images of Caenorhabditis elegans embryos, fixed and processed by immunofluorescence with S2-HA, S12-HA, C25-HA and C76-HA polypeptides in accordance with the invention, and a comparative polypeptide C8-HA, showing, for each of these polypeptides, the VHH signal (image on the left) and the DNA signal revealed with DAPI (image on the right), each line exhibiting two acquisitions of the same field;

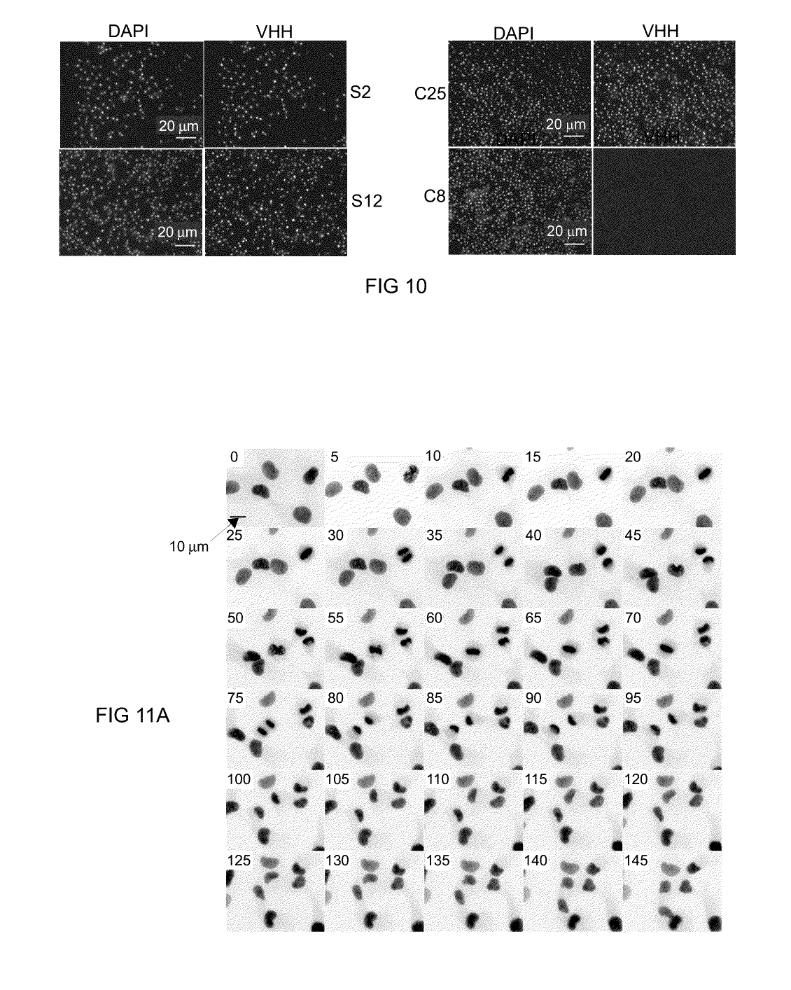

FIG. 10 shows fluorescence microscopy images of Saccharomyces cerevisiae cells processed by immunofluorescence with S2-HA, S12-HA and C25-HA polypeptides in accordance with the invention, and a comparative polypeptide C8-HA, showing, for each of these polypeptides, the VHH signal (image on the right) and the DNA signal revealed with DAPI (image on the left);

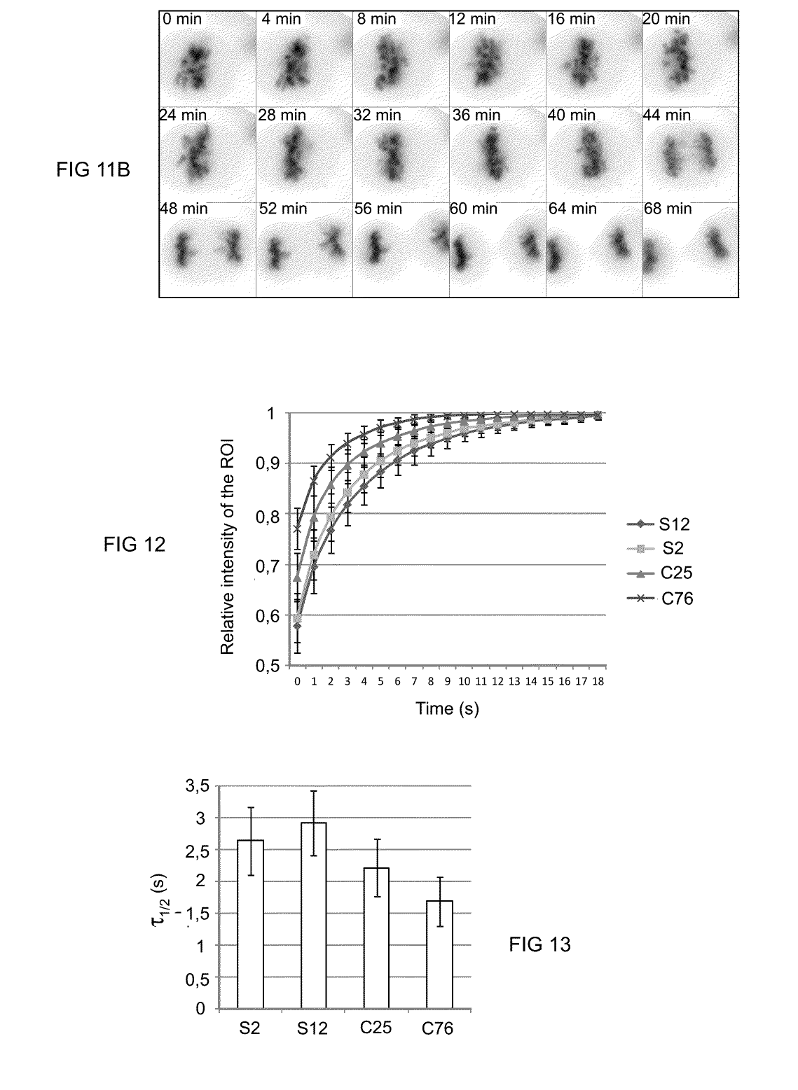

FIGS. 11A and 11B show real-time microscopy images of chromatin during the progression of the cell cycle through mytosis; FIG. 11A, real-time, wide-field microscopy images of living HT1080 cells stably expressing the S12-GFP polypeptide in accordance with the invention, a stack of images resulting from an acquisition frequency of five min being presented as an exploded view with inversion of the grayscale; FIG. 11B, fluorescence confocal microscopy images of the chromosomes of a mitotic cell expressing S12-GFP, which progresses from prometaphase to telophase, the frequency of the acquisitions presented on the exploded view being 4 min;

FIG. 12 shows the curves representing, for each of the S2-GFP, S12-GFP, C25-GFP and C76-GFP polypeptides in accordance with the invention, as a function of time, the mean (for 10 independent measurements) of the values of relative fluorescence intensity in a discrete region (ROI) of the nucleus of HT1080 cells expressing the polypeptide after photo-bleaching by laser illumination;

FIG. 13 represents a histogram presenting, for each of the S2-GFP, S12-GFP, C25-GFP and C76-GFP polypeptides in accordance with the invention, the mean (for 10 independent measurements) of the values of the fluorescence half-recovery time τ_{1/2}, in a discrete region of the nucleus of HT1080 cells expressing the polypeptide after photo-bleaching by laser illumination, these values being deduced from the adjustment of the curves formed from the relative intensity measurements as a function of time to the function I(t)=I_{E}−I₁×exp(−t/τ);

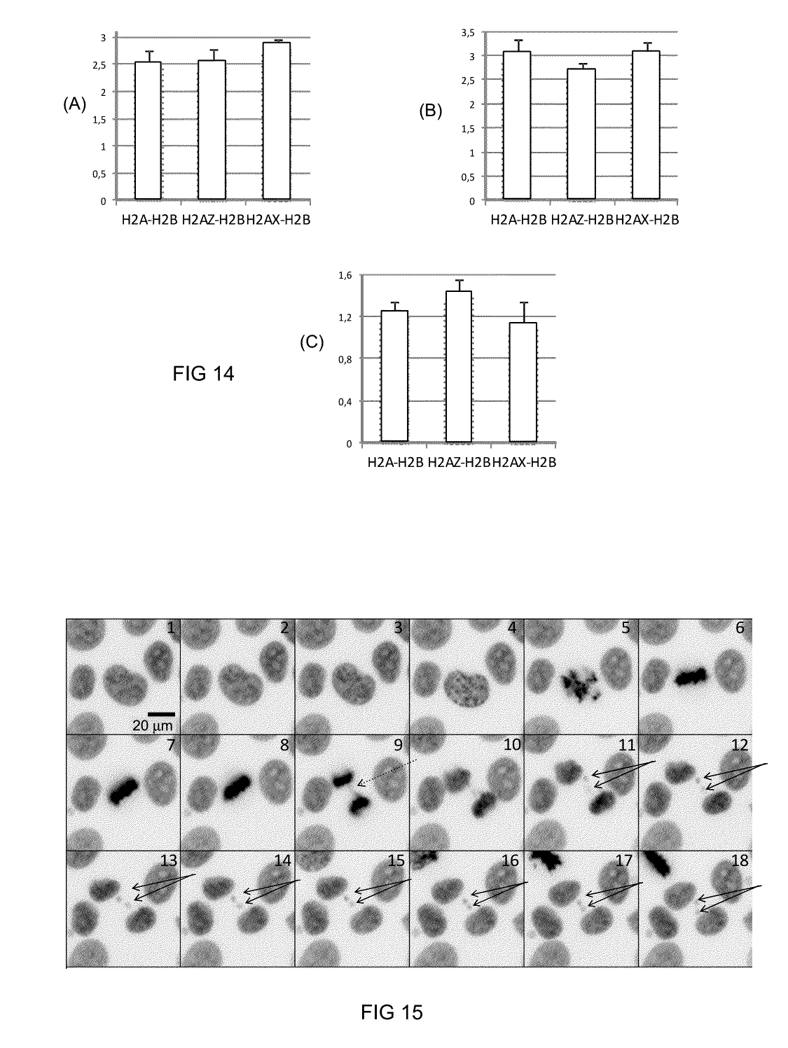

FIG. 14 shows graphs representing the optical density at 450 nm measured by an ELISA assay of binding to the human dimers respectively H2A-H2B, H2AX-H2B and H2AZ-H2B; graph (A), for the polypeptide S2 in accordance with the invention; graph (B) for the polypeptide S12 in accordance with the invention; graph (C) for a commercial anti-H2B monoclonal antibody;

FIG. 15 shows a stack of real-time, wide-field microscopy images resulting from an acquisition frequency of 10 min, of the chromatin of living HT1080 cells stably expressing the S12-GFP polypeptide in accordance with the invention during the progression of the cell cycle through mytosis, making it possible to visualize micronuclei (solid arrows) and an anaphase bridge (dashed arrow);

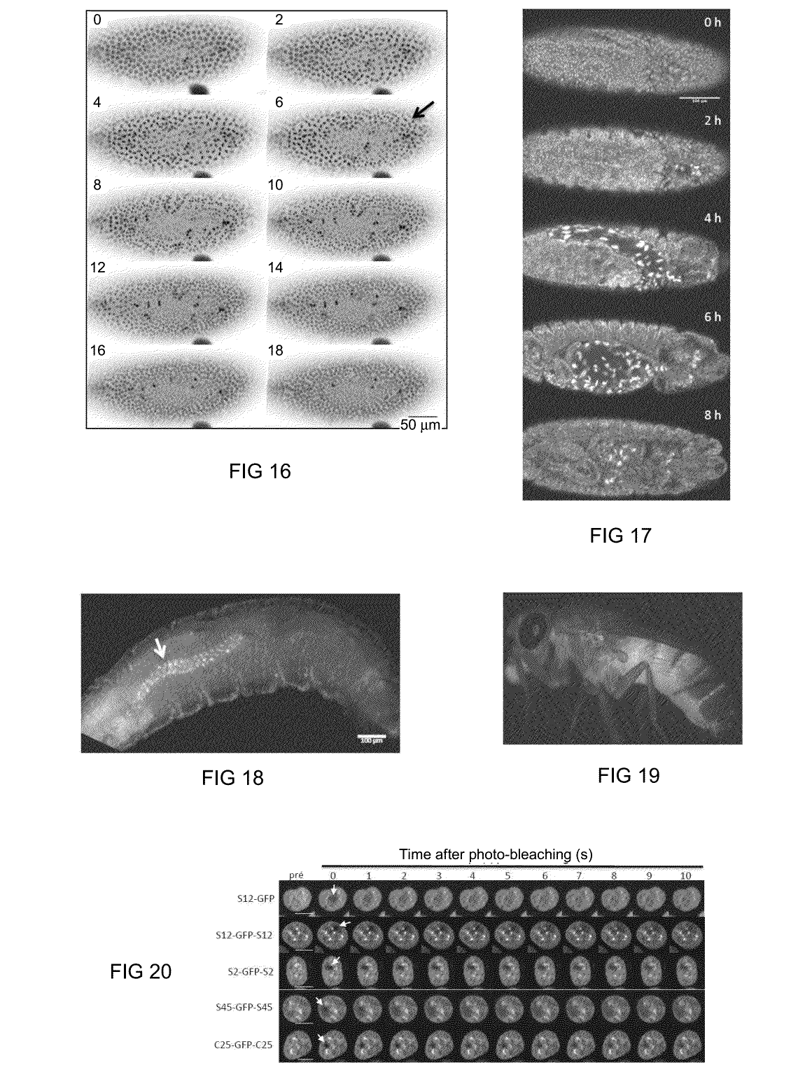

FIG. 16 shows real-time microscopy images, taken every 2 min, of a transgenic Drosophila melanogaster embryo expressing the S12-HA-GFP polypeptide in accordance with the invention at the syncytial blastoderm stage, which performs one cycle of cell division; on the image at time “6 min”, the arrow indicates anaphase figures;

FIG. 17 shows five images taken 2 h apart one after the other, originating from a real-time microscopy experiment having a total duration of 15 h, with an acquisition frequency of 2 min, of the embryo of FIG. 16;

FIG. 18 shows an epifluorescence microscopy image of a larva originating from a fluorescent embryo of FIG. 16, the arrow indicating the giant nuclei of the salivary glands;

FIG. 19 shows an image acquired by epifluorescence microscopy of an adult resulting from the metamorphosis of the larva of FIG. 18;

FIG. 20 presents confocal microscopy images acquired during FRAP experiments carried out on HCT116 human cells respectively expressing the S2-GFP-S2, S12-GFP-S12, C25-GFP-C25, S45-GFP-S45 or S12-GFP polypeptides in accordance with the invention, at various times after bleaching;

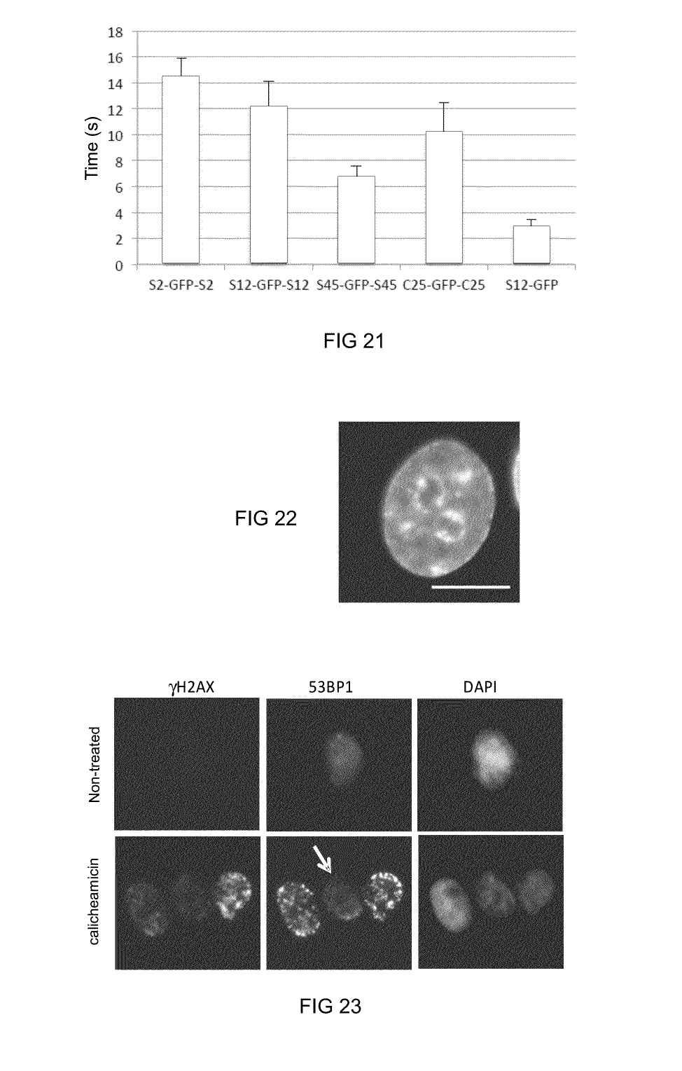

FIG. 21 represents a graph showing the fluorescence half-recovery time (T_{1/2}) after photobleaching, for the five cell types of FIG. 20;

FIG. 22 shows a confocal microscopy image of an HCT116 human cell expressing the S12-GFP-S12 polypeptide in accordance with the invention;

FIG. 23 shows the fluorescence images obtained, for a detection respectively with the anti-γH2AX and anti-53BP1 antibodies and with the DAPI intercalating agent, for HT1080 human cells which are respectively nontreated and treated with calicheamicin;

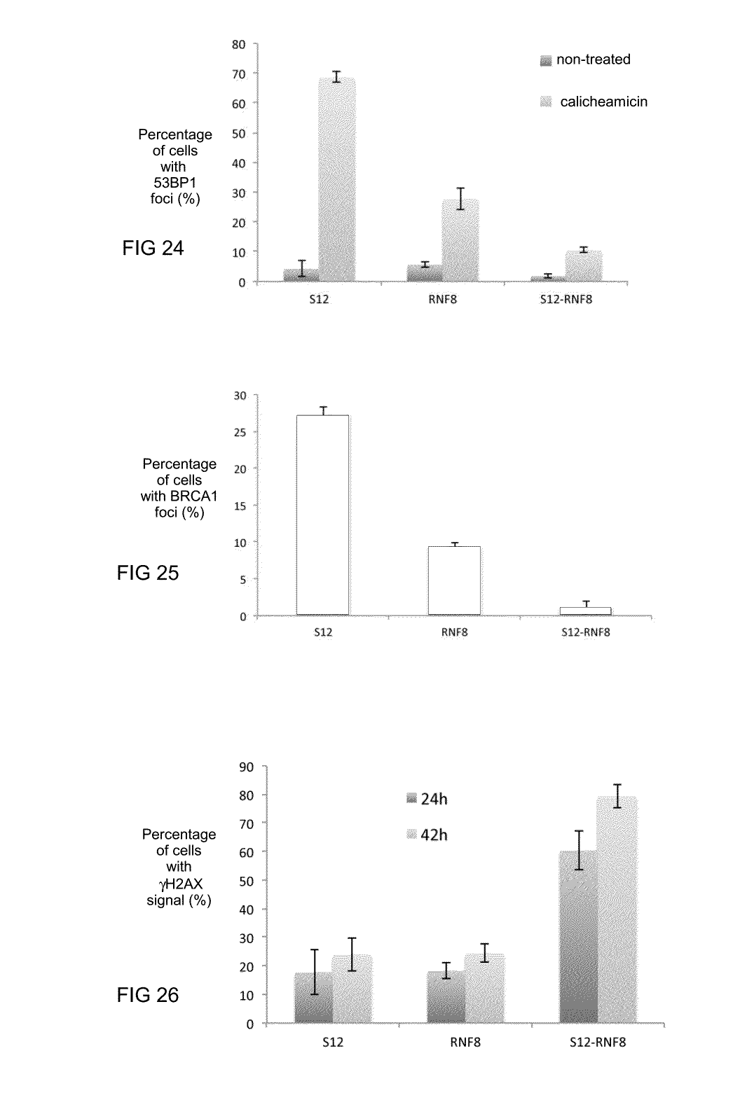

FIG. 24 represents a graph showing the percentage of HT1080 human cells, expressing either the S12-mCherry polypeptide in accordance with the invention, or the RNF8-mCherry enzyme, or the S12-mCherry-RNF8 polypeptide in accordance with the invention, exhibiting, by immunofluorescence, labeling in foci detected with the anti-53BP1 antibody, characteristic of DNA damage, with or without treatment with calicheamicin;

FIG. 25 represents a graph showing the percentage of HT1080 human cells, expressing either the S12-mCherry polypeptide in accordance with the invention, or the RNF8-mCherry enzyme, or the S12-mCherry-RNF8 polypeptide in accordance with the invention, exhibiting by immunofluorescence labeling in foci detected with the anti-BRCA1 antibody;

and FIG. 26 represents a graph showing a percentage of HT1080 human cells, expressing for 24 hours or 42 hours, either the S12-mCherry polypeptide in accordance with the invention, or the RNF8-mCherry enzyme, or the S12-mCherry-RNF8 polypeptide in accordance with the invention, exhibiting by immunofluorescence labeling in foci detected with the anti-γH2AX antibody, characteristic of DNA damage.

### EXPERIMENT 1

### Identification of Single-Domain Antibodies Directed Against Chromatin According to the Invention

Single-domain antibodies directed against chromatin according to the invention were identified by the present inventors in the following way.

### Construction of a VHH Library

An expression library dedicated to the phage display, containing the coding sequences of the VHH immune repertoire of a lama (Lama glama), was constructed. The total RNA of the lymphocytes of a lama immunized with a synthetic H2AX phosphopeptide γH2AX of sequence SEQ ID No. 6 (KKATQAS^{PO4}QEY) was isolated. It was converted to cDNA by the action of a reverse transcriptase (Superscript® II First Strand synthesis, Invitrogen), after hybridization of random primers.

The cDNAs thus generated were used as a template for a polymerase chain reaction (PCR) using the pair of primers:

CALL01:
(SEQ ID No. 7)
5′ GTCCTGGCTGCTCTTCTACAAGG 3′
CALL02:
(SEQ ID No. 8)
5′ GGTACGTGCTGTTGAACTGTTCC 3′

enabling the amplification of the variable part of the immunoglobulin heavy chains as two PCR products of different size, corresponding respectively to VH and VHH.

The VHH coding sequences were separated from the VH coding sequences by agar gel electrophoresis. After extraction of the agar gel, the VHH cDNAs were used as starting material for two successive nested PCRs, the purpose of which was to add, in the correct reading frame, the PstI cloning site in 5′ and the NotI cloning site in 3′.

After digestion with the PstI and NotI restriction enzymes, the DNA was purified and then inserted, by a ligation reaction, by means of a T4 DNA ligase, into the pHEN4 vector described in the publication by Arbabi Ghahroudi et al. (1977), digested with the same enzymes.

The ligation product was introduced into TG1 (sup E) bacteria by electroporation, and the transformed cells were plated out on 96 plates (23×23 cm) containing LB agar medium supplemented with ampicillin.

All of the bacterial colonies obtained were harvested and the diversity of the library thus generated was estimated at 6.2×10⁷.

### Establishment of Cell Lines Stably Expressing the H2AX Histone Fused to the Strep or Chitin Binding Domain (CBD) Tags

In order to be able to immobilize the H2AX histone on a solid macroscopic support (magnetic beads), vectors making it possible to establish the stable expression of two tagged forms of H2AX were constructed.

The H2AX coding sequence was amplified by PCR with the following primers:

For
(SEQ ID No. 9)
5′ ACGGTACCTCGGGCCGCGGCAAG 3′
Rev
(SEQ ID No. 10)
5′ CGGATCCCTATTAGTACTCCTGGGAGGC 3′.

The fragment generated, digested with the KpnI and BamHI restriction enzymes was then cloned, at the KpnI-BamHI sites into two expression vectors. The pCBD-H2AX vector obtained, derived from pTYB21 (New England Biolabs), allows the expression of H2AX as an N-terminal fusion with the chitin-binding domain. The p2xStrep-H2AX vector obtained, derived from Strep-tag II (iba-lifesciences), allows the expression of H2AX as an N-terminal fusion with the double Strep tag (2xStrep), of sequence SEQ ID No. 11 (WSHPQFEKGGGSGGGSGGGGSAWSHPQFEK) in which the WSHPQFEK sequence represents the Strep tag.

The cell line allowing the stable expression of CBD-H2AX was generated by transfection of the pCBD H2AX vector into HT1080 cells (human fibrosarcoma) using the jetPEI® transfection agent (PolyPlus-transfection), then selection of the stable integrants using geneticin (G418).

HT1080 cells stably expressing 2xStrep-H2AX were established by lentiviral transduction. For this, the coding sequence of the fusion was subcloned, via the NheI-BamHI sites, into the pTRIP-CMV XNCA shuttle vector (Sirven et al., 2001). The resulting pTRIP 2xStrep-H2AX vector made it possible to generate lentiviral particles, used to transduce HT1080 cells.

### Extraction of the Tagged H2AX Histone of Chromatin and Preparation of Magnetic Beads

In order to induce the phosphorylation of H2AX on serine 139, the HT1080 cells expressing CBD-H2AX or 2xStrep-H2AX previously described were treated with the radiomimetic agent calicheamicin at a final concentration of 4 nM. The histones were subsequently isolated by extraction according to the protocol described by Schechter et al. (2007). Succinctly, the nuclei were prepared by hypotonic treatment combined with the non-ionic detergent NP-40. They were lysed in a high salt buffer (2.5 M NaCl) which makes it possible to preserve the post-translational modifications and the interaction between H2A and H2B, and then the genomic DNA was fragmented by sonication. This material was used to load chitin magnetic beads (New England Biolabs) or Strep-Tactin® magnetic beads (iba-lifesciences).

To load the chitin beads, the chitin magnetic beads were washed with the washing buffer (20 mM Tris pH 8, 1 mM EDTA, 0.05% triton X-100) containing sodium chloride (NaCl) at 500 mM, and incubated in the blocking buffer (washing buffer+5% BSA (bovin serum albumin)), for 40 min at 4° C. with stirring. The beads thus blocked were washed twice with 2 M NaCl washing buffer, taken up in 1 ml of 2 M NaCl washing buffer, and then incubated with the purified histones overnight at 4° C. with stirring. The beads were then extensively washed (ten times with 2 M NaCl washing buffer), the final wash being carried out in 500 mM NaCl washing buffer. The Strep-Tactin® magnetic beads (iba-lifesciences) were loaded by incubation of the beads in a high-salt extract obtained on HT1080::2xStrep-H2AX cells, and then the material was washed in a 300 mM NaCl phosphate buffer.

### Clone Selection by Phage Display and Sublibrary Generation

This step was initially aimed at selecting VHH coding sequences having affinity for the tagged γH2AX histone, isolated from the chromatin of transgenic human cells. The selection process, described in the publication by Lee et al. (2007), involved three distinct phases. In a first step, a first sublibrary was generated by phage-display selection against CBD-γH2AX on the chitin beads, with BSA as blocking agent. This first VHH sublibrary was then used as a base in two distinct phage-display selections, according to the protocol described hereinafter: one again against CBD-γH2AX, with β-casein as blocking agent in order to generate the sublibrary termed C, the other against 2xStrep-γH2AX, in order to generate the sublibrary termed S.

### Library Amplification and Phage Generation

A bacterial aliquot of 1 ml (representing 100 times the diversity of the library) was amplified in 30 ml of TY2X medium (20 min, 37° C., 250 rpm) until an OD at 600 nm at 0.25 was obtained, before being diluted in 100 ml of TY2X supplemented with ampicillin at 100 μg/ml and with glucose at 1%. The culture was incubated for 2 h, at 37° C. and 250 rpm until an OD at 600 nm of 0.5 was obtained.

The bacteria are then infected with 10¹² helper phages, corresponding to a multiplicity of infection (MOI) of 40, and incubated for 1 h at 37° C., without shaking. After incubation for 20 min at 37° C. at 230 rpm, the culture is centrifuged for 10 min at 2000 rpm. The bacterial pallet, resuspended in 300 ml of TY2X supplemented with ampicillin at 100 μg/ml and with kanamycin at 75 μg/ml, is incubated overnight at 30° C. at 130 rpm. The culture is centrifuged for 20 min at 5000 g, and the supernatant containing the phages is recovered. The latter are precipitated for 1 h in the cold by means of 4% PEG8000/0.5 M NaCl, centrifuged for 30 min at 4000 rpm at 4° C. and then resuspended in a sterile 1×PBS solution, in a proportion of approximately 10¹² phages/ml. The suspension is incubated with 1 ml of blocking buffer (washing buffer (20 mM Tris, pH 8, 1 mM EDTA, 0.05% triton X-100) supplemented with NaCl at 500 mM and in 5% BSA) and 40 μl of beads, preequilibrated in 500 mM NaCl washing buffer, for 40 minutes at 4° C. with stirring. The phages which do not non-specifically bind to the beads are recovered in the supernatant.

### Strategy for Affinity Purification of the Phages

The phages are incubated with the CBD-γH2AX chitin beads for 3 h at 4° C. with stirring. The beads are washed extensively (10 washes (washing buffer at 500 mM NaCl), the last two washes being carried out without Triton® X-100), then incubated in washing buffer supplemented with 1.25 μg/ml trypsin and without Triton®, for 30 min at ambient temperature and with stirring, in order to inactivate the helper phages. The beads are then incubated in a 2.8% TEA solution for 7 min with stirring. After the addition of 1.5 M Tris, pH 7.4, with gentle stirring, the supernatant containing the eluted phages is recovered.

The eluted phages are used to infect TG1 bacteria, cultured in LB medium supplemented with 1% glucose, until an OD at 600 nm of 0.35 is reached, at 30° C. without shaking, for 1 h (MOI of 40). Furthermore, the TG1 bacteria are added to the beads containing non-eluted phages, and incubated at 30° C. without shaking for 1 h. The TG1 bacteria are combined and plated on TY2X agar medium supplemented with ampicillin at 100 μg/ml and with 0.5% glucose, and incubated overnight at 30° C. The colonies are harvested in liquid LB medium, and the bacterial suspension is centrifuged for 10 min at 2000 rpm and 4° C. The bacterial pallet is resuspended in order to form the sublibrary of the next round of the phage display. The addition of 50% glycerol allowed the storage thereof at −80° C.

The second round of phage display was carried out in a manner identical to that previously described, while changing however the tag of the protein immobilized on the column (2xStrep tag), the affinity beads (Strep-Tactin® beads, iba-lifesciences) and the blocking agent (β-casein, 1 mg/ml final concentration). The bacterial suspension obtained at the end of this second round of phage display represents the sublibrary S (from which the clones S2 and S12 described hereinafter are derived).

In parallel, a second strategy for affinity purification of the phages was developed. This strategy is comparable to that described previously, except for the following modifications. The first cycle of phage display was carried out on a Strep-Tactin® column prepared with the 2xStrep-γH2AX fusion protein and BSA as blocking agent. The second cycle of phage display was carried out on chitin beads with the CBD-γH2AX fusion protein and β-casein as blocking agent. Finally, the fusion proteins were obtained from the extraction of histone carried out on the stable lines previously described according to the high-salt concentration extraction protocol (Shechter et al. (2007)) followed by sonication in order to fragment the chromatin. This second strategy made it possible to obtain a sublibrary termed C (from which the clones C25 and C76 described hereinafter are derived).

### Screening of the Sublibraries S and C and Identification of Polypeptides According to the Invention

The strategy retained for evaluating the sublibraries generated consisted in clonally producing (in the periplasma) the VHHs of the sublibrary, from randomly picked bacterial clones then using the clonal bacterial medium into which the VHH-HA is secreted in immunofluorescence on fixed cells and, finally, analyzing, by fluorescence microscopy, the intracellular structure recognized by the VHH clone.

A 96-well plate (deep well) containing bacterial culture medium (2TY supplemented with 100 μg/ml ampicillin) is clonally inoculated with randomly picked clones of a sublibrary. The plate is incubated at 37° C. with shaking for 5 h. The OD₆₀₀ reaches approximately 0.4. Isopropyl β-D-1-thiogalactopyranoside (IPTG) is then added to each well (to achieve a final concentration of 1 mM), so as to induce VHH production. The culture temperature is decreased to 28° C. and the production is carried out overnight.

In parallel, a 96-well plate dedicated to cell imaging (BD Falcon® imaging plate, color is black bottom transparent) is seeded with HT1080 cells in a proportion of 2×10⁴ cells per well, and placed in a cell culture CO₂ incubator overnight. The bacterial culture plate is centrifuged (4000 rpm, 15 min) in order to separate the bacteria from their medium and to specifically transfer the supernatant containing the secreted VHH-HA. The cells in culture in the wells of the imaging plate are treated with etoposide (10 μM) for 1 h, to induce DNA breaks, are fixed with 4% paraformaldehyde for 15 min and permeabilized by treatment with 0.5% Triton® X100 for 5 min. 50 μl of VHH-HA bacterial medium from the daughter plate are transferred into a 96-well plate, the wells of which contain 50 μl of a 1/1000 dilution of an anti-HA antibody (anti-HA 11 clone 16B12 Covance). This mixture is transferred into the imaging plate containing the fixed and permeabilized cells. After incubation (1 h at ambient temperature) and washing with PBS, the anti-HA is detected by adding to the wells of the plate 50 μl of anti-mouse antibody coupled to Alexa Fluor® 488 (diluted to 1/1000 and incubated for 1 hour on the cells).

After washing with the secondary antibody, the DNA is labeled with DAPI, and then the cells of each well of the plate are imaged using an automated inverted microscope (ArrayScan Cellomics HCS reader).

The images obtained for the sublibrary S are shown in FIG. 1. In this figure, the top image represents the signal obtained by immunofluorescence, and the bottom image represents the DAPI signal. It is observed therein that remarkably specific uniform chromatin labeling is observed for a certain number of clones.

The plasmid DNA of all these clones producing a VHH which labels chromatin was purified, and then sequenced. The sequences were aligned in order to purge the redundancies. Eliminating the redundancies of the chromatin clones made it possible to reveal two VHHs of the sublibrary S, called respectively S2 (of sequence SEQ ID No. 1) and S12 (of sequence SEQ ID No. 2), and also two VHHs of the sublibrary C, called respectively C25 (of sequence SEQ ID No. 3) and C76 (of sequence SEQ ID No. 4).

The peptide sequences of the VHH domain of each of these clones are shown in FIG. 2. Indicated in this figure are the various conserved and hypervariable regions of each sequence, and also the conserved residues particularly important for binding to chromatin in the CDR3, respectively in positions 104, 105, 107 and 110 according to Kabat numbering. These amino acids, and also the other amino acids conserved between the various sequences, in CDR1, CDR2 and CDR3, are demonstrated by a box in this figure.

By way of negative comparative example for the tests hereinafter, a clone C8 of sequence SEQ ID No. 5, showing no affinity for chromatin, was also selected.

### EXPERIMENT 2

### Production of the Recombinant Polypeptides in Escherichia coli

The S2-HA, S12-HA, C25-HA and C75-HA polypeptides in accordance with the invention, and also the comparative polypeptide C8, were produced in E. coli, in the following way.

For each, the pHEN4-VHH bacterial periplasmid expression vector described above in experiment 1 is used to transform the E. coli BL21 (DE3) strain (transformation with CaCl₂).

2TY culture medium containing 100 μg/ml of ampicillin and 0.2% of glucose is inoculated with an isolated colony derived from this transformation, and then subjected to shaking at 37° C. at 220 rpm until an OD_{600 nm} of 0.5 is obtained. IPTG, at a final concentration of 1 mM, is then added to the culture, which is continued at 28° C. overnight (i.e. for 16 h). The bacterial suspension is centrifuged at 7000 rpm for 10 min, and the medium, containing the polypeptide according to the invention, is recovered.

A sample of 8 μl of each solution thus obtained is subjected to analysis by separation on a denaturing polyacrylamide gel (SDS-PAGE) and staining of the gel with Coomassie blue. Solutions of BSA at various known concentrations are subjected to the same operations, so as to evaluate, by comparison of the intensity of the respective bands, the concentration of the polypeptide according to the invention (corresponding to a single-domain antibody directed against chromatin-HA tag chimera) secreted into the medium.

The gel obtained after staining is shown in FIG. 3. For each sample of culture medium, a band of approximately 15 kDa, corresponding to the S2-HA, S12-HA, C25-HA and C75-HA polypeptides according to the invention and to the comparative polypeptide C8-HA, is observed on said gel. For each of the polypeptides tested, a concentration of polypeptide (corresponding to the band indicated by an arrow in the figure) of approximately 20 μg/ml is also deduced therefrom.

These solutions can be used directly for immunofluorescence or immunoblotting.

### EXPERIMENT 3

### Identification of the Molecular Target of the S2, S12, C25 and C76 Polypeptides in Accordance with the Invention

In order to identify the target of S2, S12, C25 and C76, these polypeptides produced in recombinant form, were used in immunoblotting experiments. The C8 polypeptide was used as a comparative control.

### 3.1/Analysis by Immunoblotting of Total Extracts of Human Cells

In order to determine whether the polypeptides which are subjects of the present invention are capable of recognizing their target immobilized on a nitrocellulose membrane, and where appropriate whether the pattern of the signal is modified by DNA damage, and in order to define the molecular weight of the target on the basis of its electrophoretic mobility, a conventional immunoblotting experiment was carried out on total protein extracts prepared from human cells treated or not treated with etoposide.

To this effect, the total cell proteins of HCT116 human cells, treated (“Eto+”) or not treated (“Eto−”) with the genotoxic etoposide, are extracted by direct lysis of the cells in the SDS loading buffer. The lysate thus obtained is sonicated before being loaded onto a gel.

The proteins are separated by electrophoresis in a denaturing polyacrylamide gel (NuPage® Bis Tris, Life Technologies), then transferred onto a nitrocellulose membrane.

After blocking in TBS-T (Tris buffered saline, 0.1% Tween® 20), 5% skimmed milk, the membrane is incubated for 1 h in a dilution of the VHH-HA polypeptide (5 μg/ml) combined with an anti-HA antibody (HA-11 mouse monoclonal 16B12 from Covance) diluted to 1 μg/ml in TBS-T, 5% skimmed milk. The membranes are also probed with the anti-H2AX (Epitomics clone EPR895) and anti-γH2AX (Millipore) monoclonal antibodies. After 3 washes in TBS-T, the blot is incubated with the anti-mouse secondary antibody coupled to horseradish peroxidase (HRP) (Cell Signaling), then washed 3 times for 10 min. Visualization is carried out with an ECL substrate (Enhanced Chemiluminescence West Dura Thermo scientific). The chemiluminescence photon emission is captured by a camera (G-Box Syngene).

The images obtained are shown in FIG. 4A. It is observed therein that the S2, S12, C25 and C76 polypeptides in accordance with the invention all recognize a band of similar electrophoretic mobility, slightly above 15 kDa. The nature of the signals generated by the polypeptides of the present invention is not modified by the induction of DNA damage, contrary to what is observed with the anti-γH2AX antibody control. Notably, the apparent molecular weight of γH2AX and those of the species recognized by the polypeptides which are subjects of the invention are similar, suggesting that said polypeptides could recognize an H2A or H2B histone.

### 3.2/Analysis by Immunoblotting on Human Cell Histones Obtained by Acid Extraction

In order to test this hypothesis, but also to determine whether the VHHs which are subjects of the invention recognize a phosphor-epitope, the histones were prepared from human cells in culture, treated or not treated with etoposide, by acid extraction.

To this effect, histones are extracted from cells cultured in vitro (HT1080), which are untreated or which have been treated with etoposide, using the acid extraction technique described in the publication by Schechter et al. (2007). Very briefly, after trypsination of a culture adherent on plastic, 10⁷ cells are washed with PBS and pre-extracted by resuspension, in 1 ml of extraction buffer (10 mM HEPES, pH 7.5, 10 mM KCl, 1.5 mM MgCl₂, 0.34 M sucrose, 0.5% NP40, 10% glycerol, protease and phosphatase inhibitor cocktail (Thermo)).

After incubation for 10 min in ice, the insoluble material (nuclei) is pelleted by centrifugation (10 000 g, 10 min at 4° C.). The nuclei are taken up in 400 μl of 0.4 N H₂SO₄ and incubated on a wheel for 2 h. After centrifugation (16 000 g, 10 min at 4° C.), the supernatant containing the histones is recovered and the histones are precipitated by adding trichloroacetic acid (TCA, 33% final concentration) and incubated on ice for 1 h. The histones are pelleted by centrifugation at 16 000 g, for 10 min at 4° C., and the pellet is washed twice in acetone cooled to −20° C. After drying, the histone pellet is taken up in 100 μl of water.

Some of the histones thus obtained by acid extraction are, if required, dephosphorylated by treatment with alkaline phosphatase (Roche) according to the protocol provided by the supplier.

The two histone preparations thus generated, treated or not treated with phosphatase, are separated by SDS-PAGE and transferred onto nitrocellulose membrane, according to the protocol described above. Staining with Ponceau red is carried out in parallel.

The results obtained are shown in FIG. 4B. It is observed therein that the S2, S12, C25 and C76 polypeptides recognize molecular species of which the mobilities are very similar and located slightly above the 15 kDa molecular marker.

Notably, the nature of the signals is modified neither by the DNA damage nor by the phosphatase treatment, whereas both of these two treatments greatly modify the signal generated by the anti-gamma H2AX antibody (used here as the dephosphorylation control).

These results show that the target of the polypeptides which are subjects of the present invention is soluble in acid, and is not a phospho-epitope. Furthermore, the superposition of the stainings with Ponceau red, which makes it possible to visualize the proteins present on the membrane, with the signal generated by the VHHs, S2, S12, C25 and C76, shows that the latter recognize molecular species located between H3/H2B (which have a virtually identical mobility under these electrophoresis conditions) and H2A. These data restrict the targets of the VHHs in accordance with the invention to the H3, H2A and H2B histones, and make it possible to exclude the H4 histone, and also the H1 histone, as a molecular target.

### 3.3/Analysis by Immunoblotting on Purified Histones

In order to precisely characterize the target of the VHHs which are subjects of the invention, the same immunoblotting experiments were carried out on the individual histones, obtained by purification by HPLC.

In order to individually purify each of the 5 classes of histones, a protocol for histone separation by reverse-phase high performance chromatography (RP HPLC), described in the article by Shechter et al. (2007), was followed. Several milligrams of histones solubilized in water were injected into a C4 reverse-phase column equilibrated with 5% of acetonitrile, 0.1% of trifluoroacetic acid (TFA) in the water. The elution of the histones was carried out by means of an acetonitrile gradient (5-90% over the course of 120 min, with a flow rate of 0.8 ml/min). The fractions obtained (each of 1 ml) were lyophilized then taken in 100 μl of water, before being analyzed by SDS-PAGE. The fractions containing the pure histones were retained, and were used to characterize the target of the polypeptides which are subjects of the invention.

To this effect, the proteins of the fractions thus obtained, in particular of the fractions containing H2A, H2B and H3, and also a mixture of the H2A and H2B fractions, are separated by electrophoresis in a denaturing polyacrylamide gel (NuPage® Bis Tris, Life Technologies), then transferred onto a nitrocellulose membrane. The visualization is carried out according to the protocol described above.

The results obtained are shown in FIG. 5. This figure shows S2, S12, C25 and C76 are capable of generating a signal only on the lane which contains the H2A-H2B mixture. This signal is precisely located at the interface of the two bands H2A and H2B, along the lines of a recognition of the H2A-H2B dimer, which became reconstituted at the interface of the two proteins deposited on the nitrocellulose membrane. The H3 histone is not recognized whereas a weak halo emanates from the lanes where the H2A and H2B histones are in the monomeric state. These results show that S2, S12, C25 and C76 do not recognize H3. On the other hand, they reveal that their target is the H2A-H2B dimer, but not the monomers.

The test above can be carried out in order to select, from the VHHs obtained by modification of the sequences SEQ ID Nos 1 to 4, those which have the capacity to bind to the H2A-H2B dimer.

### EXPERIMENT 4

### Immunofluorescence Test on Cells in Culture

### 4.1/Materials and Methods

The HCT116 line (human colon adenocarcinoma) and the MEFs (murine embryonic fibroblasts) are propagated in DMEM (Gibco Life Technologies) supplemented with 10% of fetal calf serum and antibiotics (penicillin 10 000 units/ml and streptomycin 10 000 μg/ml from Gibco Life Technologies, diluted to 1/100), and incubated in an H₂O-saturated atmosphere containing 5% of CO₂.

For each cell type, the cells cultured on glass coverslips dedicated to cell imaging are washed in a phosphate buffered saline (PBS), then fixed in a solution of PBS supplemented with 4% paraformaldehyde for 15 min.

The cells are permeabilized by incubation for 5 min in PBS supplemented with 0.25% of Triton® X100, then blocking is carried out in PBS-1% BSA.

The cells are incubated for one hour in a mixture of VHH-HA polypeptide according to the invention and 5 μg/ml and of an anti-HA antibody (Covance HA-11 mouse monoclonal, used at 0.5 μg/ml). After three washes with PBS, an anti-mouse secondary antibody coupled to Alexa Fluor® 488 (Molecular Probes Life Technologies) diluted to 1/1000 was added to the coverslips which are incubated for 45 min at ambient temperature.

The cells are washed three times in PBS, the third washing medium containing DAPI at 1 μg/ml. Finally, the mounting on a slide is carried out with Dako mounting medium.

### 4.2/on Human Cells

The polypeptides according to the invention comprising the respective antibodies S2, S12, C25 and C76 (VHH), fused to the HA tag, and a comparative polypeptide comprising the VHH C8, also fused to the HA tag, were tested on fixed HT116 human cells.

Fluorescence microscopy observation of each of the slides is carried out. The results are shown in FIG. 6. They clearly demonstrate that polypeptides in accordance with the invention comprising the antibodies S2, S12, C25 or C76, generate a chromatin signal on the fixed human cells, contrary to the comparative polypeptide C8-HA. The observation of the DNA by means of the DAPI signal is, for its part, identical for all the polypeptides. On the right of each figure, the magnification of a cell undergoing mytosis details the binding of the polypeptides to the mitotic chromosomes.

### 4.3/On Murine Cells

The polypeptides according to the invention comprising the antibodies S2, S12, C25 and C76, fused to the HA tag, were tested on fixed murine MEF cells (wild-type murine embryonic fibroblasts), treated or not treated with etoposide. The MEF cells are seeded onto glass coverslips dedicated to imaging. One day after the seeding, the cells are optionally treated for 1 h with etoposide (Sigma Aldrich) diluted in the culture medium to a final concentration of 10 μM. The cells are then fixed and permeabilized, and then incubated with a mixture of VHH-HA and anti-HA, as previously described. In order to verify the effectiveness of the induction of DNA damage by the etoposide, a coverslip is immunostained with an anti-γHA2X antibody (anti-phosphohistone H2AX Ser¹³⁹ mouse monoclonal, clone JBW301, from Millipore, diluted 1/1000 final concentration).

Fluorescence microscopy observation of each of the slides is carried out, in order to detect the signals respectively generated by the DAPI (showing the DNA), the anti-γH2AX and the polypeptides in accordance with the invention. The results are shown in FIG. 7.

The polypeptides in accordance with the invention (comprising the antibodies S2, S12, C25 or C76) generate a signal independent of the presence of DNA double breaks, contrary to the signal obtained with the anti-γH2AX immunofluorescence, which shows the double-strand break foci after treating with etoposide.

### EXPERIMENT 5

### Immunofluorescence Test on Fixed Embryos of Drosophila melanogaster

The embryos, obtained after harvesting of eggs laid by wild-type Oregon R females, were dechorionated with bleach and fixed with 4% paraformaldehyde, then stored in methanol at −20° C. The embryos were then rehydrated by means of 5 washes in PBS-0.3% Triton® X100 and incubated for 30 min in PAT (PBS, 0.3% Triton® X100 and 1% BSA). The primary antibody solution consists of a mixture of the VHH-HA polypeptide (5 μg/ml final concentration) with an anti-HA antibody (Covance HA-11 monoclonal diluted to 0.5 μg/ml) in the PAT. These primary antibodies are applied to the embryos overnight at 4° C. The latter are then washed 5 times for 10 min with PBS-0.3% Triton® X100.

The primary antibodies are visualized with an anti-mouse antibody coupled to Alexa 488 (Molecular Probe Life Technologies) diluted to 1/1000 in PAT, by incubation for 5 h. The washing of the secondary antibody is identical to that of the primary antibodies, except that the first washing is carried out in PBS-0.3% Triton® X100 containing 1 mg/ml RNase A, and 10 μg/ml of propidium iodide in order to label the DNA. The embryos are mounted in Dako mounting medium and imaged with a Zeiss LSM 510 confocal microscope.

The images obtained, for each of the S2-HA, S12-HA, C25-HA and C76-HA polypeptides in accordance with the invention, and for the comparative polypeptide C8-HA, are shown in FIG. 8. It is observed therein that, contrary to the comparative polypeptide C8-HA, the polypeptides in accordance with the invention allow very selective labeling of the chromatin.

### EXPERIMENT 6

### Immunofluorescence Test on the Caenorhabditis elegans Nematode

The Caenorhabditis elegans nematodes are collected and washed in PBS. The teguments of the animal are destructured and permeabilized using the freeze cracking technique between two glass coverslips. Thus prepared, the worms are attached to a slide treated with poly-L-lysine and fixed by incubation in an ice-cold acetone bath (5 min), followed by an ice-cold methanol bath (5 min). The fixed worms are rehydrated in PBS, before incubation for 1 h in an antibody buffer of PBS containing 0.5 mM EDTA, 0.5% Triton® X100, 1% BSA and 2% fetal calf serum.

The primary antibody mixture used is identical to that previously described with reference to experiment 5 above. The slides are incubated overnight with the primary antibody mixture, then washed 3 times for 20 min in washing buffer (PBS, 0.25% Triton® X100, 0.1% BSA).

The anti-mouse secondary antibody coupled to Alexa 488, diluted to 1/1000, is applied to the slides for 5 h. The slides are washed 3 times for 20 min in PBS, 0.25% Triton® X100, 0.1% BSA. During the second wash, the buffer contains DAPI (1 μg/ml) in order to label the DNA.

The final mounting is carried out with Dako mounting medium, and the labeled nematodes are imaged with a Zeiss LSM 510 confocal microscope.

The images obtained, for each of the polypeptides in accordance with the invention, and for the comparative polypeptide C8-HA, are shown in FIG. 9. Here again it is observed that, contrary to the comparative polypeptide C8-HA, the polypeptides in accordance with the invention allow selective labeling of the chromatin.

### EXPERIMENT 7

### Immunofluorescence Test on the Saccharomyces cerevisiae Yeast

The Saccharomyces cerevisiae yeasts of a 50 ml culture are fixed by adding 7.2 ml of 37% paraformaldehyde, and incubated for 45 min with gentle shaking. The cells are pelleted by centrifugation and washed 3 times with a PBS buffer containing 1.2 M sorbitol.

The cells are then resuspended in 1 ml of PBS containing 1.2 M sorbitol, 28 mM β-mercaptoethanol and 1 mM phenylmethylsulfonyl fluoride (PMSF). The walls are digested by adding 50 μl of 10 mg/ml zymolyase. After incubation for 20 min at 37° C., the cells are pelleted and washed with PBS containing 1.2 M sorbitol.

The suspension of spheroplasts is deposited on a glass coverslip covered with poly-L-lysine, and the cells that have adhered are fixed by adding to the coverslip 70% ethanol cooled to −20° C. The incubation in ethanol is carried out overnight at −20° C. The cells are rehydrated by incubating the coverslip in PBS.

The membranes are permeabilized by incubation for 5 min in PBS containing 0.1% NP40, 0.1% BSA. The cells are washed in PBS containing 0.1% BSA, and then the mixture of VHH-HA and anti-HA antibody diluted in PBS containing 0.1% BSA is applied to the coverslips. Incubation for 2 h with the primary antibodies is followed by washing in PBS.

The anti-mouse secondary antibody coupled to Alexa 488, diluted to 1/1000, is applied to the coverslips for 1 h. The coverslips are then washed a first time in PBS containing DAPI at a final concentration of 1 μg/ml, then a second time in PBS, before being mounted on a slide using the Dako mounting medium. The yeasts are imaged on a wide-field upright microscope (Leica DM5000), with the 63× objective.

The images obtained, for each of the S2-HA, S12-HA and C25-HA polypeptides according to the invention, and also for the comparative polypeptide C8-HA are shown in FIG. 10. Here again it is observed, contrary to the comparative peptide C8-HA, the polypeptides in accordance with the invention allow selective labeling of the chromatin.

### EXPERIMENT 8

### Real-Time Cell Imaging Test on Human Cells

The objective of this experiment was to determine whether polypeptides which are subjects of the invention, comprising a VHH fused to the GFP fluorescent protein, could be stably expressed without affecting the capacity of the cells to proliferate and also to evaluate the performance levels of these VHH-GFPs in the fluorescent labeling of the chromatin of living cells. The experiment was carried out with the S2, S12, C25 and C76 VHHs in accordance with the invention.

### Materials and Methods

### Construction of the Expression Vectors

In order to express the polypeptides comprising a VHH domain as a fusion with GFP in human cells, the coding sequence of the VHHs is amplified by PCR (with Phusion® DNA polymerase from New England Biolabs), using the following primers:

N1VHH-F:
(SEQ ID No. 12)
5′ ATACTGGAGCCACCATGGCCCAGGTGCAGCTG 3′
and
N1VHH-R:
(SEQ ID No. 13)
5′ AATGGATCCGCGTAGTCCGGAACGTCGTACG 3′

introducing the XhoI and BamHI sites in the 5′ and 3′ positions of the sequence.

The PCR fragments digested with these enzymes are cloned at the XhoI-BamHI sites of the pEGFP N1 expression vector (Clontech). In order to establish, by lentiviral transduction, cell lines stably expressing the VHH-GFP fusions, the coding sequences of the pEGFP N1 vectors were subcloned into the pTRIP lentiviral shuttle vector.

### Cell Culture and Lentiviral Transduction

The HT1080 (fibrosarcoma) and 293T (transformed embryonic kidney line) human cells are propagated in DMEM (Gibco Life Technologies) supplemented with 10% of fetal calf serum and antibiotics (penicillin and streptomycin, 10 000 U/ml from Life Technologies), and incubated in an H₂O saturated atmosphere containing 5% of CO₂.

The viral particles are produced by cotransfection (calcium chloride) of the pTRIP VHH-GFP shuttle vector with the Gag-Pol 8.91 and VSV-G vectors in 293T cells. The cell supernatants from the tritransfected cells, containing the lentiviral particles, are used, after titration, to transduce the HT1080 cells.

### Real-Time Cell Imaging

The cells expressing a VHH-GFP are seeded (20 000 to 80 000 cells per well) into culture chambers dedicated to real-time imaging (2-well Labtek, Nunc), and incubated for 24 to 72 h before imaging.

The real-time microscopy of the transgenic cells progressing in the cell cycle is carried out using a wide-field inverted microscope (Zeiss Axio Observer Z1) equipped with a CoolSnap ES² camera, with an incubation chamber at controlled atmosphere (in terms of temperature, hygrometry, CO₂ level) and with a motorized platform controlled by the Metamorph software. The cells are imaged every 5 min under transmitted light and under fluorescence using a 20× objective.

The real-time imaging of mitotic cells is likewise carried out using a laser scanning confocal microscope (Zeiss LSM 510) in the inverted position, equipped with a thermostatic incubation chamber identical to the one previously described, with a motorized platform controlled by the Zen software (Zeiss) and with a 40× water-immersion objective. The acquisition frequency was 4 min. The stacks of images generated are processed with the Fiji software.

### Results

The successions of images thus obtained are shown respectively in FIGS. 11A and 11B, for the particular example of the S12-GFP polypeptide in accordance with the invention. Similar results were obtained for the S2-GFP, C76-GFP and C25-GFP polypeptides.

Since the expression of the VHH-GFP transgenes is maintained in the transduced cells as they are successively passaged, this shows that the presence of the polypeptides which are subjects of the invention in the cell is perfectly compatible with the normal progression of the cell division cycle.

The real-time imaging of the GFP fluorescence emanating from the transgenic cells (FIG. 11A) reveals specific labeling of the chromatin in the living cells, similar to that obtained by immunofluorescence in the fixed cells. It is important to note that the localization in the nuclear compartment of the polypeptides which are subjects of the present invention is not the result of the action of a nuclear localization sequence (NLS), since the VHH-GFP fusion does not have one. On the other hand, this phenomenology is very probably linked to passive diffusion through the pores of the nuclear envelope permitted by the small size of the molecule.

As shown in FIG. 11A, the VHH-GFP fusions allow the monitoring in cell imaging, on the scale of a cell population or of the single cell, of the cell motility, of the shape of the nucleus, of the shape of the nucleoli, and of the structure of the chromatin.

Furthermore, the polypeptides in accordance with the invention thus expressed in the cells allow the real-time monitoring of the choreography of the mitotic chromosomes during cell division (FIG. 11B), allowing in particular the study of a defect of chromatin condensation, of chromatid segregation in anaphase, of metaphase plate rotation, and of the duration of the various phases of mitosis. The polypeptides according to the invention can thus advantageously be used for HCS (high content screening) of libraries of compounds, or in RNAi.

### Detection of Micronuclei and Anaphase Bridges

Clearer images, after processing using the image J software, obtained with the S12-GFP polypeptide, are shown in FIG. 15. In this figure, the successive images correspond to acquisitions carried out every 10 min.

It is observed therein that, during mitosis, the chromosomes align on the metaphase plate (images 6-7-8), then separate into two equivalent batches in the daughter cells (image 9). The polypeptide in accordance with the invention makes it possible to observe the presence of two micronuclei (solid arrow, images 11-18), for 1 h 20, clearly indicating the presence of cytogenetic lesions. A finer analysis also makes it possible to observe an anaphase bridge (dashed arrow, image 9), characteristic of a mitosis defect.

These results show that the polypeptide in accordance with the invention makes it possible to monitor mitosis defects (anaphase bridges), but also cytogenetic lesions, of micronuclei type. This polypeptide therefore makes it possible to carry out genotoxicity tests requiring the visualization of chromatin, such as the micronuclei test.

### EXPERIMENT 9

### Test of Mobility of the Polypeptides on their Target in Living Cells

The objective of this experiment was to evaluate and quantifier, by means of an approach of photo-bleaching of the GFP carried out on the VHH-GFP transgenic cells, the mobility of S2-GFP, S12-GFP, S45-GFP, C25-GFP and C76-GFP polypeptides which are subjects of the present invention on their histone target in the nucleus of living cells.

### Methodology

The fluorescence recovery after photo-bleaching (FRAP) experiments were carried out on the HT1080 cells which stably express the various VHH-GFPs above. The optical tool for photo-bleaching, fluorophore excitation and photon-emission acquisition is a laser scanning confocal microscope (Zeiss LSM 510) in the inverted position, equipped with a thermostatic incubation chamber identical to the one previously described, and controlled by the Zen software (Zeiss). The objective used is a 40× water-immersion objective.

The photo-bleaching and the fluorophore excitation are carried out using an argon laser tuned to 488 nm. A circular subregion of the nucleus (region of interest: ROI) is arbitrarily defined. The illumination-acquisition fluorescence FRAP sequence consists of a first acquisition (initial state), then an illumination with the argon laser (488 nm) of the ROI resulting in photo-bleaching of the zone is triggered, directly followed by an acquisition (t=0) which takes place every second until a stabilized return of the fluorescence emitted. For each polypeptide, the FRAP measurements were carried out on at least 10 independent cells.

The calculations of the mean intensity of the ROI, its correction, the relative mean (mean intensity/maximum value of the fluorescence return signal), the establishment of the curve of the relative intensity of the ROI as a function of time, then the adjustment of this curve to a mathematical relationship (#) linking the intensity as a function of time (making it possible to deduce the value of the half-recovery time τ_{1/2}) are carried out by the macro FRAP of the Zen software. The calculation of the means and standard deviations (from independent experiments for each polypeptide) of the values of relative intensity as a function of time and of the deduced τ_{1/2} values, and the establishment of the related curves and histogram were carried out using the Excel spreadsheet.

(*)l(t)=lg−l₁×exp(−t/τ)

Where I_{E} is the value at the recovery plateau, I₁ is the value of the mobile fraction, τ is the time constant which describes the mobility of the molecule studied, τ_{1/2} is the fluorescence half-recovery time of the photo-bleached fluorescent molecule with τ_{1/2}=In0.5/−τ.

### Results

The curves of fluorescence recovery as a function of time, for each polypeptide in accordance with the invention, are shown in FIG. 12.

The values of the deduced half-recovery time τ_{1/2} of the polypeptide on its target, for each of the polypeptides tested, are presented in FIG. 13.

The τ_{1/2} value scale, between 3 and 1.5 seconds, indicates a relatively high mobility of the polypeptides in accordance with the invention on their histone target.

### EXPERIMENT 10

### Test for Specificity with Respect to the Major Dimers of H2A-H2B

The specificity of binding of the S2 and S12 polypeptides in accordance with the invention to the H2AX-H2B and H2AZ-H2B dimers was studied by an ELISA approach.

The human H2A, H2AX, H2AZ and H2B histones were obtained by coexpression of the H2A-H2B dimer in the E. coli bacterium, followed by two steps of purification by affinity chromatography, according to a procedure described in the publication by Anderson et al. (2010).

The HA-tagged and His₆-tagged VHHs were produced by secretion in the E. coli bacteria. The polypeptide was purified from the bacterial medium by ammonium sulfate precipitation followed by affinity chromatography on a cobalt resin.

For the ELISA assay, the three types of dimers H2A-H2B, H2AX-H2B and H2AZ-H2B were diluted in PBS to 1 μg/100 μl, then 100 μl of each dimer dilution were distributed in duplicate in the wells of a Maxisorp® 96-well plate (Nunc). The adsorption of the histone dimers to the plastic was carried out overnight at 4° C. The wells were blocked using 100 μl of PBS+10% of skimmed milk for 2 h. The wells were then washed using 100 μl of PBS+0.1% Tween® 20 (PBST). 100 μl of PBST+5% of skimmed milk containing the purified HA-tagged VHH polypeptide diluted to 1 ng/ml and an anti-HA antibody diluted to 0.5 ng/ml were added to the wells.

An anti-H2B antibody (rabbit monoclonal antibody (D2H6), Cell Signaling #12364) was used as both a positive control and a control for loading with the various wells.

After incubation at ambient temperature for 1 h, the wells were washed with 100 μl of TBST, and 100 μl of anti-mouse secondary antibody coupled to HRP, diluted to 1/5000 in TBST, were added to the wells. The plate was incubated for 45 min, then two washes using 100 μl/well of TBST were carried out. The addition of 100 μl of TMB substrate (Sigma Aldrich) made it possible to visualize the HRP activity associated with the secondary antibody. The intensity of the signal in each well was quantified by reading the OD_{450 nm} in a plate reader (Multiskan®). The analysis of the optical density (OD) values, and in particular the calculation of the means, was carried out using the Excel spreadsheet.

The results obtained are shown in FIG. 14.

Graphs A and B show that the S2 and S12 polypeptides bind the three types of H2A-H2B dimer in a not significantly different manner. The similar signal intensities between the wells of the three histone dimers obtained with the commercial anti-H2B control (graph C) show, moreover, that the three types of histone dimers are present in equivalent amount in the wells of the experimental device.

### EXPERIMENT 11

### Imaging of the Chromatin of a Model Transgenic Drosophila Organism

A transgenic drosophila ubiquitously expressing the S12-HA-GFP polypeptide in accordance with the invention was generated.

### Materials and Methods

### Cloning of the Expression Vector

In order to be able to carry out the site-specific insertion, into the genome of the fly Drosophila melanogaster, of a cassette enabling the expression of S12-GFP under the UAS-Gal4 expression system (Phelps et al. (1998)), the coding sequence of the S12-HA-GFP fusion obtained from the pEGFPN1 S12-HA construct (described in experiment 8) was cloned, using the XhoI-XbaI restriction sites, into the pUAS dedicated transgenesis vector (Bischof et al. (2007)).

### Generation of the Transgenic Flies and Expression of the Transgene

The insertion of the S12-HA-GFP transgene, locus 68A4 of chromosome 3, was carried out by microinjection of the pUAS S12-HA-GFP plasmid into the germ cells of embryos (nosC31NLS; attP2) which express the phiC31 integrase (Bischof et al. (2007)). The ubiquitous expression of the transgene was carried out by means of an actin or ubiquitin driver.

### Live Fluorescence Imaging of Drosophila

For imaging of the live embryo, the eggs were manually dechlorionated before being placed in a drop of oil (Halocarbon) on a microscope slide. The imaging of the fluorescence was carried out using a 25× objective of a confocal microscope (Zeiss LSM 510) equipped with a chamber thermoregulated at 25° C. For the real-time imaging of the embryonic development, the acquisitions were carried out every 2 min. The imaging of the larvae, and of the live adult flies expressing S12-HA-GFP was carried out using a Macrofluo® microscope (Leica) equipped for epifluorescence illumination of the sample, and provided with a CoolSNAP® ES² camera. The images generated were processed using the image J software.

### Results

The confocal imaging of the fluorescence emitted by live embryos expressing the S12-HA-GFP polypeptide was carried out. Chromatin-specific fluorescent labeling is observed.

FIG. 16 shows real-time microscopy images, taken every 2 min, of an embryo at the syncytial blastoderm stage which is performing a cell division cycle (synchronous at this stage of development). It is observed therein that the S12-HA-GFP probe polypeptide makes it possible to visualize the synchronous mitosis at this moment of embryonic development. The image at time “0 min” shows how the S12-HA-GFP probe makes it possible to visualize the interphase nuclei. In the image at time “4 min”, the metaphase plates are recognizable, and in the image at time “6 min”, the probe makes it possible to clearly visualize anaphase figures (indicated by an arrow in the right-hand part of the embryo). In the images acquired later (time “10 min”), doubling of the number of nuclei can be clearly observed.

FIG. 17 shows five images taken 2 h apart one after the other, originating from a real-time microscopy experiment lasting a total of 15 h, with an acquisition frequency of 2 min, of an embryo expressing the S12-HA-GFP probe. These images show that the VHH-GFP fusion remains specifically linked to the chromatin throughout embryonic development, and demonstrate the performance levels of the tool in the visualization, over long periods of time, of chromatin.

The larvae resulting from these fluorescent embryos were observed by epifluorescence microscopy. The image obtained was shown in FIG. 18. The fluorescence is specifically concentrated in the nuclei, which can be observed by transparency. In particular, the giant nuclei of the salivary glands are clearly visible, and indicated by an arrow on the figure.

FIG. 19 shows an image acquired by epifluorescence microscopy of an adult resulting from the metamorphosis of a larva expressing the S12-HA-GFP probe. This adult is perfectly alive and it continues to express the transgene.

These results of imaging carried out during the development of a drosophila ubiquitously expressing the S12-GFP polypeptide in accordance with the invention demonstrate that this polypeptide specifically labels chromatin in an invertebrate, this being in a manner similar to what could be imaged in human cells. In addition, the polypeptide remains linked to the chromatin throughout development up to the adult stage. The expression of the polypeptide is not invasive, i.e. it does not prevent development of the drosophila from the embryonic stage to the adult stage. These results clearly show that the polypeptide in accordance with the invention can be used to visualize chromatin with high spatial and temporal resolution in extremely dynamic cell processes.

### EXPERIMENT 12

### Diabodies

Polypeptides in accordance with the invention of use for chromatin imaging were generated by concatenation of VHH around GFP.

### Materials and Methods

### Cloning of the Fluorescent “Diabody” Constructs

In order to place a VHH domain on either side of GFP, the strategy was to clone the NheI-BsrG1 fragment, originating from the pEGFPN1 VHH vector, at the NheI-BsrG1 sites of the pEGFPC1 VHH vector. The new expression vectors thus generated relate to the following VHHs: S2, S12, S45 and C25. They were called respectively pEGFP S2-GFP-S2, pEGFP S12-GFP-S12, pEGFP S45-GFP-S45 and pEGFP C25-GFP-C25.

Cell expression and study of the dynamics of the probes by analysis of the speed of fluorescence recovery after photo-bleaching (FRAP).

In order to express the diabody fusions in human cells and to carry out the real-time imaging thereof, HCT116 cells were transfected with the pEGFP VHH-GFP-VHH vectors (using the JetPEI® transfection agent) in 2-well Lab-Tek® format (Nunc). The imaging of the living transfected cells, and the FRAP experiments, were carried out with a 40× immersion objective of a confocal microscope (Zeiss LSM 510). The sequence of the operations for the FRAP was as follows:

1) acquisition pre-photobleaching,
2) illumination of a defined region of the nucleus in the form of a circle, resulting in quenching of the emission of fluorescence in the zone,
3) the photo-bleaching sequence was immediately followed by an acquisition sequence with a frequency of 1 s for 1 min.

The establishment of the intensity values corrected on the basis of the raw data of mean intensity in the photo-bleach region was carried out using the FRAP module of the Zen software (Zeiss). This software corrects the raw intensity data so as to take into account the photo-bleaching linked to the acquisition sequence, and the background noise. The fluorescence half-recovery time (T_{1/2}) was calculated by the software by agreement with a monoexponential mathematical function:

l(t)=l_{E}−l₁×exp(−t/τ)

in which I_{E} is the value at the recovery plateau, I₁ is the value of the mobile fraction, τ is the time constant which describes the mobility of the molecule studied, τ_{1/2} is the fluorescence half-recovery time of the fluorescent molecule with τᵥ₂=In0.5/−τ.

The graphs were established using the Excel spreadsheet. The images were processed by image J.

### Results

FIG. 20 presents montages of images from FRAP experiments carried out on HCT116 cells expressing the VHH-GFP-VHH fusions, but also the S12-GFP construct as a control representative of a probe which has only one VHH module. Under these image acquisition rate conditions, it is noted that the circle-shaped quenching in the nucleus resulting from the photo-bleaching is clearly visible with the “diabody” probes, which is symptomatic of a lower mobility. On the other hand, under the same experiment conditions, this photo-bleaching appears to be diffuse with the S12-GFP probe, a phenomena linked to a considerable dynamic. Furthermore, visually, the fluorescence recovery is slower in the “diabody” probes compared with S12-GFP. Finally, visual examination of the montages also makes it possible to see differences between the four VHH-GFP-VHH constructs in the recovery speed.

The quantification of the intensities made it possible to establish fluorescence recovery kinetics curves, for a mean calculated on nine independent cells for each probe. These quantifications clearly show that the fluorescence recovery is slower with the “diabody” probes, in comparison with the mono-VHH control probe S12-GFP. The half-recovery time (T_{1/2}) was calculated from each fluorescence recovery curve. The mean of the values established for each construct is presented in FIG. 21. The graph shows therein that, generally, the “diabody” probes have a significantly longer half-recovery time than the mono-VHH probe S12-GFP. In particular, the τ_{1/2} of the “diabody” version of S12 is four times longer than that of S12-GFP. The graph also shows a considerable disparity between the “diabody” probes, the S2-GFP-S2 probe being the least mobile, while S45-GFP-S45 is the most mobile of the “diabody” constructs.

Most of the “diabodies”, and in particular those comprising S2 and S12, also make it possible to visualize nuclear compartments that the mono-VHH polypeptides do not reveal, as can be seen in FIG. 20. FIG. 22 shows a representative image of what makes it possible to visualize the S12-GFP-S12 probe in the nucleus of a human cell. This signal pattern—peri-nucleolar and peri-nuclear labeling—is symptomatic of a probe which preferentially recognizes heterochromatin.

These results demonstrate that the polypeptides according to the invention combining two VHH modules in the same peptide chain exhibit less mobility of the chromatin than the polypeptides comprising a single VHH module. These polypeptides constitute unique tools for visualizing in real time, in living cells, the dynamics of the heterochromatin domains.

### EXPERIMENT 13

### Modification of the Chromatin State-Immunofluorescence Test on Cells in Culture

The objective of this experiment is to demonstrate that the coupling of an activity to the polypeptides which are subjects of the present invention makes it possible to modify the overall state of chromatin.

To this effect, a polypeptide is generated in which S12 is coupled to the RNF8 protein, which is an E3 ubiquitin ligase specifically recruited to sites of DNA double-stranded breaks (DSBs), where it is essential for ubiquitinylation of the H2A and H2AX histones. The locally ubiquitinylated H2A and H2AX histones allow the recruitment to the sites of DSBs of the 53BP1 and BRCA1 proteins, two key factors in DSB repair (Mailand et al. (2007)).

The S12-RNF8 construct, and also the S12 and RNF8 proteins alone, were transiently expressed in human cells in order to verify whether the recruitment of 53BP1 and BRCA1 was impaired when VHH S12 is fused to RNF8.

### Materials and Methods

### Construction of the Expression Vectors

The coding sequence of VHH S12 was subcloned into a eukaryotic expression vector as a C-terminal fusion with mCherry fluorescent protein, so as to form the plasmid called pAO-S12-mCherry.

The coding sequence of RNF8 previously inserted into the pEGFP-C1 vector (Mailand et al. (2007)) was subcloned into the pmCherry-C1 vector so as to form the plasmid called pmCherry-RNF8.

The coding sequence of mCherry-RNF8 originating from the pmCherry-RNF8 plasmid was subcloned into the pEGFP-N1 vector containing the coding sequence of VHH S12 (described in experiment 8), in place of the coding sequence of EGFP, so as to form the plasmid called pS12-mCherry-RNF8.

### Cell Culture and Imaging

The HT1080 line (human fibrosarcoma) was propagated in DMEM medium (Gibco Life Technologies) supplemented with 10% of fetal calf serum and antibiotics (penicillin 10 000 units/ml and streptomycin 10 000 μg/ml from Gibco Life Technologies, diluted to 1/100), and incubated in an H₂O-saturated atmosphere containing 5% of CO₂.

The various plasmids were transfected by means of the TranslT®-2020 reagent (Mirus) into the cells placed in culture on glass coverslips dedicated to cell imaging, in order to allow the transient expression of the constructs for 24 to 42 h. According to the experiment, the cells were treated for 1 h with 10 picoM of calicheamicin (Wyeth), so as to induce DSB formation, before being washed in phosphate buffer saline (PBS), then fixed in a solution of PBS supplemented with 4% paraformaldehyde, for 20 min.

The cells were permeabilized by incubation for 15 min in PBS supplemented with 0.5% Triton® X100, then blocking was carried out in PBS-10% goat serum.

The cells were incubated for 1 h with either an anti-53BP1 antibody (rabbit polyclonal NB100-304, Novus Biologicals, used at 0.33 μg/ml), or an anti-BRCA1 antibody (mouse monoclonal sc-6954, Santa Cruz Biotechnology, used at 2 μg/ml), or anti-γH2AX antibody (mouse monoclonal JBW301, Merck/Millipore, used at 0.33 μg/ml). After three washes with PBS, an anti-mouse or anti-rabbit secondary antibody coupled to Alexa Fluor® 488 (Molecular Probes Life Technologies), diluted to 1/1000, was added to the coverslips, which were incubated for 45 min at ambient temperature.

The cells were washed 3 times in PBS, the third washing medium containing DAPI at 1 μg/ml. Finally, the mounting on a slide was carried out with Dako mounting medium. The fluorescence imaging of the cells was carried out as previously described.

### Results

FIG. 23 shows the images obtained, for detection respectively with the anti-γH2AX and anti-53BP1 antibodies and with the DAPI intercalating agent, for cells respectively not treated and treated with calicheamicin.

It is observed therein that the DSBs can be visualized in the cell through the observation of various biomarkers such as γH2AX (form phosphorylated on serine 139 of the H2AX histone variant) and 53BP1. In the absence of treatment, the cells do not experience any DSBs and exhibit no γH2AX signal, whereas 53BP1 is found diffusively throughout the nucleus (FIG. 23 at the top). The calicheamicin treatment, by inducing DSBs, results in phosphorylation of H2AX in all the cells, resulting in a predominantly punctate γH2AX labeling (in the form of foci), which labels the DSB sites (FIG. 23 at the bottom). 53BP1 also forms foci which colocalize with the γH2AX foci. In some cells, this labeling does not appear in the form of foci (white arrow), due to an excessive number of DSBs or to a 53BP1 recruitment defect.

The proportion of cells exhibiting 53BP1 foci in response to calicheamicin was calculated, for the cells expressing respectively the S12, RNF8 and S12-RNF8 polypeptides. The results obtained are shown in FIG. 24. It is observed therein that, when the cells express VHH S12, the proportion of cells exhibiting 53BP1 foci in response to calicheamicin is close to 70%. However, this proportion drops to 28% when the cells express RNF8, and to 10% when the cells express S12-RNF8.

The proportion of cells exhibiting BRCA1 foci, in DNA replication S phase, was also calculated, for the cells expressing respectively S12, RNF8 and S12-RNF8 polypeptides. The results obtained are shown in FIG. 25. It is observed therein that 27% of the cells expressing S12 exhibit BRCA1 foci, which represents the expected proportion of cells in S phase for cells in culture. On the other hand, only 9% and 1% of cells expressing respectively RNF8 and S12-RNF8 show BRCA1 foci.

In order to verify that the cells expressing the S12-RNF8 construct exhibit a DSB repair defect, the γH2AX signal was analyzed in greater detail. FIG. 26 shows the proportion of cells showing a γH2AX signal, for the cells expressing respectively the S12, RNF8 and 512-RNF8 polypeptides, for expression times of 24 h and 42 h. It appears that, even in the absence of exogenous damage, a strong proportion of cells expressing S12-RNF8 exhibits a γH2AX signal, compared with the cells expressing S12 or RNF8. Furthermore, this proportion of γH2AX-positive cells increases with the construct expression time. This result indicates that the cells which express S12-RNF8 accumulate unrepaired DSBs over time.

The above results show that the polypeptides in accordance with the invention comprising VHH S12 make it possible to modify the function of a protein by modifying its localization. In the present case, RNF8 fused to S12 can no longer be specifically localized at the DSB sites, since the VHH continually directs it onto the whole of the chromatin. As a result, the ubiquitin ligase function of RNF8 is diluted to the whole of the chromatin, and therefore can no longer be enriched at the DSB sites. This results in an inability of the 53BP1 and BRCA1 proteins to be normally recruited to the DSBs, and therefore a defect in terms of repair of these lesions, which explains the accumulation of the γH2AX signal. The polypeptides according to the invention can therefore be used to alter the general state of chromatin, by diversion of the specific recruitment of chromatin-modifying proteins.

### LITERATURE REFERENCES

Anderson et al. (2010) Protein expression and purification 72(2): 194-204
Arbabi Ghahroudi et al. (1997) FEBS Lett., 414(3): 521-6
Bischof et al. (2007) Proc. Natl. Acad. Sci. U.S.A. 104: 3312-3317
Chiu et al. (2010) Molecular Pharmacology, 77(4): 497-507
Kirsch-Volders (1997) Mutation Res., 392: 1-4
Lee et al. (2007) Nature Protocols, 2(11): 3001-8
Mailand et al. (2007) Cell, 131(5): 887-900
Phelps et al. (1998) Methods, 14: 367-379
Scechter et al. (2007) Nature Protocols, 2(6): 1445-57
Sirven et al. (2001) Mol Ther., 3(4): 438-48

1-21. (canceled)

22. A polypeptide comprising a single-domain antibody directed against chromatin, derived from a heavy-chain antibody naturally devoid of a light chain (VHH) of a camelid and capable of binding specifically to a complex of H2A and H2B histones.

23. A polypeptide as claimed in claim 22, wherein said single-domain antibody has the amino acid sequence of any one of the sequences SEQ ID No. 1, SEQ ID No. 2, SEQ ID No. 3 or SEQ ID No. 4.

24. A polypeptide as claimed in claim 22, wherein said single-domain antibody has a sequence comprising one or more deletion(s), addition(s) or substitution(s) of one or more amino acids with respect to one of the sequences SEQ ID No. 1, SEQ ID No. 2, SEQ ID No. 3 or SEQ ID No. 4, which do not significantly modify the binding characteristics of the antibody to a complex of H2A and H2B histones.

25. A polypeptide as claimed in claim 22, wherein said single-domain antibody has a functional portion of one of the sequences SEQ ID No. 1, SEQ ID No. 2, SEQ ID No. 3 or SEQ ID No. 4 which retains the binding site(s) and the protein domain(s) required for binding to a complex of H2A and H2B histones.

26. A polypeptide as claimed in claim 22, wherein said single-domain antibody is a VHH domain.

27. A polypeptide as claimed in claim 22, wherein said single-domain antibody comprises, in position 107 according to Kabat numbering, an arginine residue.

28. A polypeptide as claimed in claim 22, wherein said single-domain antibody comprises, in position 110 according to Kabat numbering, a serine residue or a threonine residue.

29. A polypeptide as claimed in claim 22, wherein said single-domain antibody comprises, in position 104 according to Kabat numbering, a glycine residue.

30. A polypeptide as claimed in claim 22, wherein said single-domain antibody comprises, in position 105 according to Kabat numbering, a serine residue or a tyrosine residue.

31. A polypeptide as claimed in claim 22, comprising a plurality or single-domain antibodies directed against chromatin, each of said antibodies being derived from a heavy-chain antibody naturally devoid of a light chain (VHH) of a camelid and being capable of binding specifically to a complex of H2A and H2B histones.

32. A polypeptide as claimed in claim 22, also comprising a peptide sequence corresponding to a functional peptide/protein of interest.

33. A polypeptide as claimed in claim 32, wherein said peptide/protein of interest is a detectable protein.

34. A polypeptide as claimed in claim 22, also comprising a peptide sequence corresponding to a cell-penetrating peptide.

35. A nucleic acid molecule encoding a polypeptide as claimed in claim 22.

36. An expression vector comprising a nucleic acid molecule as claimed in claim 35.

37. A host cell comprising a nucleic acid molecule as claimed in claim 35.

38. A transgenic non-human animal expressing a polypeptide as claimed in claim 22.

39. A method of using a polypeptide as claimed in claim 22 for detecting and/or visualizing chromatin in real time.

40. A method as claimed in claim 39, wherein said polypeptide comprises a peptide sequence corresponding to a functional peptide/protein of interest and a peptide sequence corresponding to a cell-penetrating peptide, and wherein said functional protein of interest is a protein detectable by fluorescence, luminescence or phosphorescence, for visualizing chromatin in real time in living cells.

41. A method of using a polypeptide as claimed in claim 22 for visualizing chromatin mitotic profile disruptions.

42. A method of using a polypeptide as claimed in claim 22 for modifying chromatin fibers in vitro, on fixed cells or on living cells in culture.

43. A method of using a polypeptide as claimed in claim 22 for purifying chromatin.

44. A kit comprising a polypeptide as claimed in claim 22, and/or a nucleic acid molecule encoding said polypeptide, and/or a vector comprising said nucleic acid molecule and/or a host cell comprising said nucleic acid molecule.


SEQUENCE LISTING (13 sequences)

SEQ ID NO: 1 - protein, 125 aa, Lama glama
MAQVQLQASGGGLVQAGDSLRLSCAAAGRSFSSYAMGWFRQAPGKEREFVAAISRSRGVTYADSVKGRFTISKDNVKNMAYLQMNSLKPEDTGVYYCAATSSGSTRQLSTGRSNYWGQGTQVTVS

SEQ ID NO: 2 - protein, 123 aa, Lama glama
MAQVQLQASGGGLVQAGGSLRLSCAASGRTVATLGWFRQAPGKEREFVAGITWTAGRTYYADSVKGRFTISRDNKRTTYLQMNSLKPEDTGVYYCAATSSGSTRLLSTGRDNYWGQGTLVTVS

SEQ ID NO: 3 - protein, 122 aa, Lama glama
MAQVQLQASGGGLVQAGESLTLSCDASGRTFSIGAMGWFRQAPGKEREFLAAINWNGGYTSYADVASGRFTISRNAKNTVYLQMNSLQPEDTAVYYCAAKYGGSARSRTYDYWGQGTQVTVS

SEQ ID NO: 4 - protein, 121 aa, Lama glama
MAQVQLQASGGGLVQAGGSLRLSCAVSGGTLSSYTMGWFRQAPGKEREFVSALSRVGGKSDYADSVKGRFTISRNAKNTVYLQMNSLKPEDTAVYYCAAGGYSRRWSAYNYWGQGTQVTVS

SEQ ID NO: 5 - protein, 127 aa, Lama glama
MAEVQLQASGGGLVQPGNSLRLSCVGSGVSLSAYNMGWFRQAPGKEREFVADINVSGGTAYYADSVKGRFTISRDNVKNTLYLQMNSLKLDDAAVYYCAAAPAWRPFTYDSRDDYPYWGQGTQVTVS

SEQ ID NO: 6 - protein, 10 aa, Artificial sequence
  Phospho-peptide  H2AX synthitique
KKATQASQEY

SEQ ID NO: 7 - DNA, 23 bp, Artificial sequence
  Amorce PCR CALL01
gtcctggctgctcttctacaagg

SEQ ID NO: 8 - DNA, 23 bp, Artificial sequence
  Amorce PCR CALL02
ggtacgtgctgttgaactgttcc

SEQ ID NO: 9 - DNA, 23 bp, Artificial sequence
  Amorce PCR For
acggtacctcgggccgcggcaag

SEQ ID NO: 10 - DNA, 28 bp, Artificial sequence
  Amorce PCR Rev
cggatccctattagtactcctgggaggc

SEQ ID NO: 11 - protein, 30 aa, Artificial sequence
  Tag 2xStrep
WSHPQFEKGGGSGGGSGGGGSAWSHPQFEK

SEQ ID NO: 12 - DNA, 32 bp, Artificial sequence
  Amorce PCR N1VHH-F
atactcgagccaccatggcccaggtgcagctg

SEQ ID NO: 13 - DNA, 31 bp, Artificial sequence
  Amorce PCR N1VHH-R
aatggatccgcgtagtccggaacgtcgtacg